(19) United States
(12) Patent Application Publication
(10) Pub. No.: US 2025/0236579 A1
(43) Pub. Date: Jul. 24, 2025

(54) METHOD FOR EXTRACTING FLUORINE-CONTAINING ETHER COMPOUND

(71) Applicant: AGC INC., Tokyo (JP)

(72) Inventor: Takuya IWASE, Tokyo (JP)

(73) Assignee: AGC INC., Tokyo (JP)

(21) Appl. No.: 19/173,893

(22) Filed: Apr. 9, 2025

Related U.S. Application Data
(63) Continuation of application No. PCT/JP2023/036912, filed on Oct. 11, 2023.

(30) Foreign Application Priority Data
Oct. 13, 2022 (JP) 2022-164649

(51) Int. Cl. C07C 41/38

(52) U.S. Cl. CPC C07C 41/38

(57) ABSTRACT

To provide a method for extracting a fluorinated ether compound by which a desired fluorinated ether compound can be extracted efficiently. The method for extracting a fluorinated ether compound of the present invention is a method for extracting a fluorinated ether compound, which comprises extracting a first component from a composition with an organic solvent containing no fluorine atom, the composition containing the first component composed of at least one fluorinated ether compound selected from the group consisting of a compound represented by the formula (A1), a compound represented by the formula (A2) and a compound represented by the formula (A3), and a second component composed of at least one fluorinated ether compound selected from the group consisting of a compound represented by the formula (B1) and a compound represented by the formula (B2).

### TECHNICAL FIELD

The present invention relates to a method for extracting a fluorinated ether compound.

### BACKGROUND ART

Fluorinated ether compounds having a perfluoropolyether chain, which have high lubricity etc., are used for various applications. Methods for extracting such a fluorinated ether compound from a composition containing the fluorinated ether compound and other components, have been studied.

For example, Patent Document 1 discloses a method of extracting a perfluoropolyether oil (a fluorinated ether compound) contained in grease, with use of liquid or supercritical carbon dioxide.

### PRIOR ART DOCUMENTS

### Patent Documents

Patent Document 1: JP-A-2017-503651

### DISCLOSURE OF INVENTION

### Technical Problem

Technique to extract a fluorinated ether compound with use of liquid or supercritical carbon dioxide has been known as disclosed in Patent Document 1. However, methods for efficiently extracting a desired fluorinated ether compound by means of another method have been required.

Under these circumstances, the object of the present invention is to provide a method for extracting a fluorinated ether compound by which a desired fluorinated ether compound can efficiently be extracted.

### Solution to Problem

The present inventors have conducted extensive studies on the above object and as a result found that when extracting a desired fluorinated ether compound from a plurality of fluorinated ether compounds, the desired fluorinated ether compound can efficiently be extracted with use of an organic solvent containing no fluorine atom, and accomplished the present invention.

That is, the present inventors have found that the above object can be achieved by the following construction.
[1] A method for extracting a fluorinated ether compound, which comprises extracting a first component from a composition, using an organic solvent containing no fluorine atom, the composition containing:
   the first component composed of at least one fluorinated ether compound selected from the group consisting of a compound represented by the formula (A1), a compound represented by the formula (A2) and a compound represented by the formula (A3), and
   a second component composed of at least one fluorinated ether compound selected from the group consisting of a compound represented by the formula (B1) and a compound represented by the formula (B2),
   [R^{f1}—(OR^{f11})_{y1}—O—R¹]ⱼ-L¹-(R¹¹-T¹¹)ₓ₁ (A1)
   (T³¹-R³¹)ₓ₃-L³-R³—(OR^{f12})_{y2}—O—R²-L²-(R²¹-T²¹)ₓ₂ (A2)
   Q¹[-(OR^{f13})_{y3}—O—R⁴-L⁴-(R⁴¹-T⁴¹)ₓ₄]ᵣ₁ (A3)
   R^{f14}—(OR^{f15})_{y4}—O—R⁵—R^{b1} (B1)
   R^{f19}—(OR^{f18})_{y6}—O—R⁷-L^{b1}-R⁶—O—(R^{f16}O)_{y5}—R^{f17} (B2)
   in the formula (A1),
   R^{f1} is a C₁₋₂₀ fluoroalkyl group, and when there is a plurality of R^{f1}, the plurality of R^{f1} may be the same or different from each other,
   R^{f11} is a C₁₋₆ fluoroalkylene group, and when there is a plurality of R^{f11}, the plurality of R^{f11} may be the same or different from each other,
   R¹ is an alkylene group or a fluoroalkylene group, and when there is a plurality of R¹, the plurality of R¹ may be the same or different from each other,
   L1 is a single bond or a (j+x1)-valent group which may have N, O, S or Si and may have a branch point, and in which atoms bonded to R¹ and R¹¹ are each independently a N, O, S, Si atom, a carbon atom constituting the branch point, or a carbon atom having a hydroxy group or an oxo group (═O),
   R¹¹ is an alkylene group in which an atom bonded to L1 may be an etheric oxygen atom, and which may have an etheric oxygen atom between carbon atoms, and when there is a plurality of R¹¹, the plurality of R¹¹ may be the same or different from each other,
   T¹¹ is —CH═CH₂ or —SiR^{a11}_{z11}R^{a12}_{3-z11}, and when there is a plurality of T¹¹, the plurality of T¹¹ may be the same or different from each other,
   in a case where T¹¹ is —CH═CH₂, at least one R¹¹ has a carbon chain having 2 or more carbon atoms linked to each other, and the carbon chain is linked to T¹¹,
   in a case where T¹¹ is —SiR^{a11}_{z11}R^{a12}_{3-z11}, at least one R¹¹ has a carbon chain having 4 or more carbon atoms linked to one another,
   R^{a11} is a hydroxy group or a hydrolysable group, and when there is a plurality of R^{a11}, the plurality of R^{a11} may be the same or different from each other,
   R^{a12} is a non-hydrolysable group, and when there is a plurality of R^{a12}, the plurality of R^{a12} may be the same or different from each other,
   j is an integer of 1 or more, and when j is 1, y1 is an integer of 2 or more, or R¹ is a fluoroalkylene group,
   z11 is an integer of 1 to 3,
   x1 is an integer of 1 or more, and
   1 is an integer of 1 or more, and when there is a plurality of y1, the plurality of y1 may be the same or different from each other,
   in the formula (A2),
   R^{f12} is a C₁₋₆ fluoroalkylene group, and when there is a plurality of R^{f12}, the plurality of R^{f12} may be the same or different from each other,
   R² and R³ are each independently an alkylene group or a fluoroalkylene group,
   L² is a single bond or a (1+x2)-valent group which may have N, O, S or Si and may have a branch point, and in which atoms bonded to R² and R²¹ are each independently a N, O, S, Si atom, a carbon atom constituting the branch point, or a carbon atom having a hydroxy group or an oxo group (═O),
   L3 is a single bond or a (1+x3)-valent group which may have N, O, S or Si and may have a branch point, and in which atoms bonded to R³ and R³¹ are each independently a N, O, S, Si atom, a carbon atom constituting the branch point, or a carbon atom having a hydroxy group or an oxo group (═O),
   R²¹ is an alkylene group in which an atom adjacent to L² may be an etheric oxygen atom, and which may have an etheric oxygen atom between carbon atoms, and when there is a plurality of R²¹, the plurality of R²¹ may be the same or different from each other,
   R³¹ is an alkylene group in which an atom adjacent to L³ may be an etheric oxygen atom, and which may have an etheric oxygen atom between carbon atoms, and when there is a plurality of R³¹, the plurality of R³¹ may be the same or different from each other,
   T²¹ and T³¹ are each independently —CH═CH₂ or —SiR^{a21}_{z21}R^{a22}_{3-z21}, when there is a plurality of T²¹, the plurality of T²¹ may be the same or different from each other, and when there is a plurality of T³¹, the plurality of T³¹ may be the same or different from each other,
   in a case where T²¹ is —CH═CH₂, at least one R²¹ has a carbon chain having 2 or more carbon atoms linked to each other, and the carbon chain is linked to T²¹,
   in a case where T²¹ is —SiR^{a21}_{z21}R^{a22}_{3-z21}, at least one R²¹ has a carbon chain having 4 or more carbon atoms linked to one another,
   in a case where T³¹ is —CH═CH₂, at least one R³¹ has a carbon chain having 2 or more carbon atoms linked to each other, and the carbon chain is linked to T³¹,
   in a case where T³¹ is —SiR^{a21}_{z21}R^{a22}_{3-z21}, at least one R³¹ has a carbon chain having 4 or more carbon atoms linked to one another,
   R^{a21} is a hydroxy group or a hydrolysable group, and when there is a plurality of R^{a21}, the plurality of R^{a21} may be the same or different from each other,
   R^{a22} is a non-hydrolysable group, and when there is a plurality of R^{a22}, the plurality of R^{a22} may be the same or different from each other,
   z21 is an integer of 1 to 3,
   x2 and x3 are each independently an integer of 1 or more, and
   y2 is an integer of 1 or more, and when y2 is 1, R² is a fluoroalkylene group,
   in the formula (A3),
   Q¹ is a r1-valent group having a branch point,
   R^{f13} is a C₁₋₆ fluoroalkylene group, and the plurality of R^{f13} may be the same or different from each other,
   R⁴ is each independently an alkylene group or a fluoroalkylene group, and the plurality of R⁴ may be the same or different from each other,
   L⁴ is a single bond or a (1+x4)-valent group which may have N, O, S or Si and may have a branch point, and in which atoms bonded to R⁴ and R⁴¹ are each independently a N, O, S, Si atom, a carbon atom constituting the branch point, or a carbon atom having a hydroxy group or an oxo group (═O), and the plurality of L⁴ may be the same or different from each other,
   R⁴¹ is an alkylene group in which an atom adjacent to L⁴ may be an etheric oxygen atom, and which may have an etheric oxygen atom between carbon atoms, and the plurality of R⁴¹ may be the same or different from each other,
   T⁴¹ is —CH═CH₂ or —SiR^{a41}_{z41}R^{a42}_{3-z41}, and the plurality of T⁴¹ may be the same or different from each other,
   in a case where T⁴¹ is —CH═CH₂, at least one R⁴¹ has a carbon chain having 2 or more carbon atoms linked to each other, and the carbon chain is linked to T⁴¹,
   in a case where T⁴¹ is —SiR^{a41}_{z41}R^{a42}_{3-z41}, at least one R⁴¹ has a carbon chain having 4 or more carbon atoms linked to one another,
   R^{a41} is a hydroxy group or a hydrolysable group, and the plurality of R^{a41} may be the same or different from each other,
   R^{a42} is a non-hydrolysable group, and when there is a plurality of R^{a42}, the plurality of R^{a42} may be the same or different from each other,
   z41 is an integer of 1 to 3,
   x4 is an integer of 1 or more, and when there is a plurality of x4, the plurality of x4 may be the same or different from each other,
   y3 is an integer of 1 or more, and when there is a plurality of y3, the plurality of y3 may be the same or different from each other, and
   r1 is 3 or 4,
   in the formula (B1),
   R^{f14} is a C₁₋₂₀ fluoroalkyl group,
   R^{f15} is a C₁₋₆ fluoroalkylene group, and when there is a plurality of R^{f15}, the plurality of R^{f15} may be the same or different from each other,
   R⁵ is an alkylene group which may have a substituent,
   R^{b1} is a hydrogen atom, a chlorine atom, a bromine atom, or an iodine atom, and
   4 is an integer of 1 or more,
   in the formula (B2),
   R^{f17} and R^{f19} are each independently a C₁₋₂₀ fluoroalkyl group,
   R^{f16} and R^{f18} are each independently a C₁₋₆ fluoroalkylene group, when there is a plurality of R^{f16}, the plurality of R^{f16} may be the same or different from each other, and when there is a plurality of R^{f18}, the plurality of R^{f18} may be the same or different from each other,
   R⁶ and R⁷ are each independently an alkylene group which may have a substituent,
   L^{b1} is a single bond or a bivalent linking group (excluding (OR^{f18})_{y7} and (R^{f16}O)_{y8}, y7 and y8 are each independently an integer of 1 or more), and
   y5 and y6 are each independently an integer of 1 or more.
[2] The fluorinated ether compound extraction method according to [1], wherein the organic solvent is a non-halogenated solvent.
[3] The fluorinated ether compound extraction method according to [2], wherein the non-halogenated solvent has a relative permittivity of 30 or less.
[4] The fluorinated ether compound extraction method according to any one of [1] to [3], wherein the organic solvent is a hydrocarbon-based solvent.
[5] The fluorinated ether compound extraction method according to any one of [1] to [4], wherein in the formula (A1), in a case where T¹¹ is —CH═CH₂, at least one R¹¹ has a carbon chain having 4 or more carbon atoms linked to one another and the carbon chain is linked to T¹¹, and in the formula (A1), in a case where T¹¹ is —SiR^{a11}_{z11}R^{a12}_{3-z11}, at least one R¹¹ has a carbon chain having 6 or more carbon atoms linked to one another,
   in the formula (A2), in a case where T²¹ is —CH═CH₂, at least one R²¹ has a carbon chain having 4 or more carbon atoms linked to one another and the carbon chain is linked to T²¹, and in the formula (A2), in a case where T²¹ is —SiR^{a21}_{z21}R^{a22}_{3-z21}, at least one R²¹ has a carbon chain having 6 or more carbon atoms linked to one another,
   in the formula (A2), in a case where T³¹ is —CH═CH₂, at least one R³¹ has a carbon chain having 4 or more carbon atoms linked to one another and the carbon chain is linked to T³¹, and in the formula (A2), in a case where T³¹ is —SiR^{a21}_{z21}R^{a22}_{3-z21}, at least one R³¹ has a carbon chain having 6 or more carbon atoms linked to one another, and
   in the formula (A3), in a case where T⁴¹ is —CH═CH₂, at least one R⁴¹ has a carbon chain having 4 or more carbon atoms linked to one another and the carbon chain is linked to T⁴¹, and in the formula (A3), in a case where T⁴¹ is —SiR^{a41}_{z41}R^{a42}_{3-z41}, at least one R⁴¹ has a carbon chain having 6 or more carbon atoms linked to one another.
[6] The fluorinated ether compound extraction method according to any one of [1] to [5], wherein the group represented by (OR^{f11})_{y1} has a molecular weight of 2,000 to 20,000,
   the group represented by (OR^{f12})_{y2} has a molecular weight of 2,000 to 20,000, and
   the group represented by (OR^{f13})_{y3} has a molecular weight of 2,000 to 20,000.
[7] The fluorinated ether compound extraction method according to any one of [1] to [6], wherein
   a ratio of a molecular weight of the group represented by [R^{f1}—(OR^{f11})_{y1}—O—R¹]ⱼ to a molecular weight of the group represented by L¹-(R¹¹-T¹¹)ₓ₁ is 13% or more,
   a ratio of a molecular weight of the group represented by R³—(OR^{f12})_{y2}—O—R² to a sum of a molecular weight of the group represented by L²-(R²¹-T²¹)ₓ₂ and a molecular weight of the group represented by (T³¹-R³¹)ₓ₃-L³ is 13% or more, and
   a ratio of a sum of a sum of molecular weights of r1 pieces of groups represented by (OR^{f13})_{y3}—O—R⁴ and a molecular weight of the group represented by Q¹, to a sum of molecular weights of r1 pieces of groups represented by L⁴-(R⁴¹-T⁴¹)ₓ₄, is 13% or more.
[8] The fluorinated ether compound extraction method according to any one of [1] to [7], wherein
   in the formula (B1), R⁵ is a fluoroalkylene group, and
   in the formula (B2), both R⁶ and R⁷ are a fluoroalkylene group, and L^{b1} is a single bond.
[9] The fluorinated ether compound extraction method according to [8], wherein
   in the formula (B1), a number of carbon atoms in the fluoroalkylene group represented by R⁵ is 1 to 20, and
   in the formula (B2), a total number of carbon atoms in the fluoroalkylene group represented by R⁶ and the fluoroalkylene group represented by R⁷ is 2 to 40.

### Advantageous Effects of Invention

According to the present invention, provided is a method for extracting a fluorinated ether compound by which the desired fluorinated ether compound can efficiently be extracted.

### DESCRIPTION OF EMBODIMENTS

In this specification, a compound represented by the formula (A1) may sometimes be referred to as Compound (A1). The same applies to compounds etc. represented by other formulae.

A fluoroalkyl group generically means a perfluoroalkyl group and a partial fluoroalkyl group. The perfluoroalkyl group means an alkyl group in which all hydrogen atoms are replaced with fluorine atoms. The partial fluoroalkyl group means an alkyl group in which 1 or more hydrogen atoms are replaced with fluorine atoms and which has 1 or more hydrogen atoms. That is, the fluoroalkyl group is an alkyl group having 1 or more fluorine atoms.

A “reactive silyl group” generically means a hydrolysable silyl group and a silanol group (Si—OH), and the “hydrolysable silyl group” means a group capable of forming a silanol group when hydrolyzed.

An “organic group” manes a hydrocarbon group which may have a substituent and which may have a hetero atom or another bond in a carbon chain.

A “hydrocarbon group” is a group composed of carbon atoms and hydrogen atoms and is an aliphatic hydrocarbon group (for example, a bivalent aliphatic hydrocarbon group may be a linear alkylene group, an alkylene group having a branch or a cycloalkylene group), an aromatic hydrocarbon group (for example, a bivalent aromatic hydrocarbon group may be a phenylene group) or a combination thereof.

A “surface layer” means a layer to be formed on the surface of a substrate.

A “molecular weight” of a fluoropolyether chain is a number average molecular weight calculated from the number (average) of oxyfluoroalkylene units based on the terminal groups by means of ¹H-NMR and ¹⁹F-NMR.

A “molecular weight” of another partial structure of the fluorinated ether compound other than the fluoropolyether chain may be calculated by conducting structural analysis of the fluorinated ether compound by means of ¹H-NMR and ¹⁹F-NMR.

A range of numerical values represented by “to” means a range including numerical values before and after it as the lower limit value and the upper limit value. In the numerical ranges described stepwise in this specification, the upper limit value or the lower limit value described in a certain numerical range may be replaced with an upper limit value or a lower limit value of another numerical range described stepwise. In the numerical ranges described in this specification, the upper limit value or the lower limit value described in a certain numerical range may be replaced with a value indicated in Examples.

In this specification, each component may be used singly or in combination of 2 or more types of substances corresponding to the component. In a case where 2 or more types of substances are used in combination for a component, the content of the component means the total content of the substances used in combination, unless otherwise specified.

In this specification, a combination of 2 or more preferred embodiments corresponds to a more preferred embodiment.

### Extraction Method

The method for extracting a fluorinated ether compound of the present invention (hereinafter sometimes referred to as “present extraction method”) is a method of extracting a first component from a composition (hereinafter sometimes referred to as “present composition”) containing the first component composed of at least one fluorinated ether compound selected from the group consisting of a compound represented by the formula (A1) described later, a compound represented by the formula (A2) described later and a compound represented by the formula (A3) described later, and
a second component composed of at least one fluorinated ether compound selected from the group consisting of a compound represented by the formula (B1) described later and a compound represented by the formula (B2) described later,
with use of an organic solvent containing no fluorine atom (hereinafter sometimes referred to as a “non-fluorinated organic solvent”).

According to the present extraction method, the first component can efficiently be extracted from the present composition. The detailed mechanism for this reason is unclear, but is estimated as follows. That is, compatibility of the first component and the non-fluorinated organic solvent is improved by the number of linked carbon atoms at the R¹¹-T¹¹ moiety in the formula (A1), the number of linked carbon atoms at the R²¹-T²¹ moiety and the T³¹-R³¹ moiety in the formula (A2), and the number of linked carbon atoms at the R⁴¹-T⁴¹ moiety in the formula (A3) being predetermined values.

### Present Composition

The present composition contains the first component and the second component and may contain another component. In the following, the components contained in the present composition will be described.

### <First Component>

The first component is at least one fluorinated ether compound selected from the group consisting of a compound represented by the following formula (A1), a compound represented by the following formula (A2) and a compound represented by the following formula (A3), and is a compound to be extracted in the present extraction method.
[R^{f1}—(OR^{f11})_{y1}—O—R¹]ⱼ-L¹-(R¹¹-T¹¹)ₓ₁ (A1)
(T³¹-R³¹)ₓ₃-L³-R³—(OR^{f12})_{y2}—O—R²-L²-(R²¹-T²)ₓ₂ (A2)
Q¹[-(OR^{f13})_{y3}—O—R⁴-L⁴-(R⁴¹-T⁴¹)ₓ₄]ᵣ₁ (A3)

In the formula (A1),
R^{f1} is a C₁₋₂₀ fluoroalkyl group, and when there is a plurality of R^{f1}, the plurality of R^{f1} may be the same or different from each other,
R^{f11} is a C₁₋₆ fluoroalkylene group, and when there is a plurality of R^{f11}, the plurality of R^{f11} may be the same or different from each other,
R¹ is an alkylene group or a fluoroalkylene group, and when there is a plurality of R¹, the plurality of R¹ may be the same or different from each other,
L¹ is a single bond or a (j+x1)-valent group which may have N, O, S or Si and may have a branch point, and in which atoms bonded to R¹ and R¹¹ are each independently N, O, S, Si, a carbon atom constituting the branch point, or a carbon atom having a hydroxy group or an oxo group (═O),
R¹¹ is an alkylene group in which an atom bonded to L¹ may be an etheric oxygen atom, and which may have an etheric oxygen atom between carbon atoms, and when there is a plurality of R¹¹, the plurality of R¹¹ may be the same or different from each other,
T¹¹ is —CH═CH₂ or —SiR^{a11}_{z11}R^{a12}_{3-z11}, and when there is a plurality of T¹¹, the plurality of T¹¹ may be the same or different from each other,
in a case where T¹¹ is —CH═CH₂, at least one R¹¹ has a carbon chain having 2 or more carbon atoms linked to each other, and the carbon chain is linked to T¹¹,
in a case where T¹¹ is —SiR^{a11}_{z11}R^{a12}_{3-z11}, at least one R¹¹ has a carbon chain having 4 or more carbon atoms linked to one another,
R^{a11} is a hydroxy group or a hydrolysable group, and when there is a plurality of R^{a11}, the plurality of R^{a11} may be the same or different from each other,
R^{a12} is a non-hydrolysable group, and when there is a plurality of R^{a12}, the plurality of R^{a12} may be the same or different from each other,
j is an integer of 1 or more, and when j is 1, y1 is an integer of 2 or more, or R¹ is a fluoroalkylene group,
z11 is an integer of 1 to 3,
x1 is an integer of 1 or more, and
y1 is an integer of 1 or more, and when there is a plurality of y1, the plurality of y1 may be the same or different from each other.

In the formula (A2),
R^{f12} is a C₁₋₆ fluoroalkylene group, and when there is a plurality of R^{f12}, the plurality of R^{f12} may be the same or different from each other,
R² and R³ are each independently an alkylene group or a fluoroalkylene group,
L² is a single bond or a (1+x2)-valent group which may have N, O, S or Si and may have a branch point, and in which atoms bonded to R² and R²¹ are each independently N, O, S, Si, a carbon atom constituting the branch point, or a carbon atom having a hydroxy group or an oxo group (═O),
L³ is a single bond or a (1+x3)-valent group which may have N, O, S or Si and may have a branch point, and in which atoms bonded to R³ and R³¹ are each independently N, O, S, Si, a carbon atom constituting the branch point, or a carbon atom having a hydroxy group or an oxo group (═O),
R²¹ is an alkylene group in which an atom adjacent to L² may be an etheric oxygen atom, and which may have an etheric oxygen atom between carbon atoms, and when there is a plurality of R²¹, the plurality of R²¹ may be the same or different from each other,
R³¹ is an alkylene group in which an atom adjacent to L³ may be an etheric oxygen atom, and which may have an etheric oxygen atom between carbon atoms, and when there is a plurality of R³¹, the plurality of R³¹ may be the same or different from each other,
T²¹ and T³¹ are each independently —CH═CH₂ or —SiR^{a21}_{z21}R^{a22}_{3-z21}, when there is a plurality of T²¹, the plurality of T²¹ may be the same or different from each other, and when there is a plurality of T³¹, the plurality of T³¹ may be the same or different from each other,
in a case where T²¹ is —CH═CH₂, at least one R²¹ has a carbon chain having 2 or more carbon atoms linked to each other, and the carbon chain is linked to T²¹,
in a case where T²¹ is —SiR^{a21}_{z21}R^{a22}_{3-z21}, at least one R²¹ has a carbon chain having 4 or more carbon atoms linked to one another,
in a case where T³¹ is —CH═CH₂, at least one R³¹ has a carbon chain having 2 or more carbon atoms linked to each other, and the carbon chain is linked to T³¹,
in a case where T³¹ is —SiR^{a21}_{z21}R^{a22}_{3-z21}, at least one R³¹ has a carbon chain having 4 or more carbon atoms linked to one another,
R^{a21} is a hydroxy group or a hydrolysable group, and when there is a plurality of R^{a21}, the plurality of R^{a21} may be the same or different from each other,
R^{a22} is a non-hydrolysable group, and when there is a plurality of R^{a22}, the plurality of R^{a22} may be the same or different from each other,
z21 is an integer of 1 to 3,
x2 and x3 are each independently an integer of 1 or more, and
y2 is an integer of 1 or more, and when y2 is 1, R² is a fluoroalkylene group.

In the formula (A3),
Q¹ is a r1-valent group having a branch point,
R^{f13} is a C₁₋₆ fluoroalkylene group, and the plurality of R^{f13} may be the same or different from each other,
R⁴ is each independently an alkylene group or a fluoroalkylene group, and the plurality of R⁴ may be the same or different from each other,
L4 is a single bond or a (1+x4)-valent group which may have N, O, S or Si and may have a branch point, and in which atoms bonded to R⁴ and R⁴¹ are each independently a N, O, S, Si atom, a carbon atom constituting the branch point, or a carbon atom having a hydroxy group or an oxo group (═O), and the plurality of L⁴ may be the same or different from each other,
R⁴¹ is an alkylene group in which an atom adjacent to L⁴ may be an etheric oxygen atom, and which may have an etheric oxygen atom between carbon atoms, and the plurality of R⁴¹ may be the same or different from each other,
T⁴¹ is —CH═CH₂ or —SiR^{a41}_{z41}R^{a42}_{3-z41}, and the plurality of T⁴¹ may be the same or different from each other,
in a case where T⁴¹ is —CH═CH₂, at least one R⁴¹ has a carbon chain having 2 or more carbon atoms linked to each other, and the carbon chain is linked to T⁴¹,
in a case where T⁴¹ is —SiR^{a41}_{z41}R^{a42}_{3-z41}, at least one R⁴¹ has a carbon chain having 4 or more carbon atoms linked to one another,
R^{a41} is a hydroxy group or a hydrolysable group, and the plurality of R^{a41} may be the same or different from each other,
R^{a42} is a non-hydrolysable group, and when there is a plurality of R^{a42}, the plurality of R^{a42} may be the same or different from each other,
z41 is an integer of 1 to 3,
x4 is an integer of 1 or more, and the plurality of x4 may be the same or different from each other,
y3 is an integer of 1 or more, and the plurality of y3 may be the same or different from each other, and
r1 is 3 or 4.

Since the first component has the fluoropolyether chain, a surface layer formed by using the first component is excellent in water/oil repellency and fingerprint stain removability.

A first component having —CH═CH₂ at its terminal is suitably used as a material of a compound having a reactive silyl group at its terminal.

Since a reactive silyl group is firmly chemically bonded to a substrate, a surface layer formed by using a first component having a reactive silyl group at its terminal is excellent in durability such as abrasion resistance.

Now, structures of the respective compounds will be described. Reference symbols representing the same structures are indicated as such and may properly be read for reference.

The first component has a fluoropolyether chain, which is a group having 2 or more oxyfluoroalkylene units.

Such a fluoropolyether chain may have a hydrogen atom. In order that an obtainable surface layer will be more excellent in abrasion resistance and fingerprint removability, the proportion of fluorine atoms represented by the following formula (I) in the fluoropolyether chain is preferably 60% or more, more preferably 80% or more, further preferably substantially 100%, that is the fluoropolyether chain is a perfluoropolyether chain. When the proportion of fluorine atom is 60% or more, the fluorine amount in the fluoropolyether chain is large, and lubricity and fingerprint stain removability will more improve.

Formula (I): proportion of fluorine atoms (%)=(number of fluorine atoms)/{(number of fluorine atoms)+(number of hydrogen atoms)}×100

The molecular weight of one of the above fluoropolyether chains (that is, the molecular weight of the group represented by (OR^{f11})_{y1}, the molecular weight of the group represented by (OR^{f12})_{y2}, and the molecular weight of the group represented by (OR^{f13})_{y3}) is, in order that the obtainable surface layer has both fingerprint stain removability and lubricity, preferably 2,000 to 20,000, more preferably 2,500 to 15,000, further preferably 3,000 to 10,000. When the molecular weight of the fluoropolyether chain is 2,000 or more, the fluoropolyether chain has improved flexibility and in addition, the fluorine amount in the molecule is large, whereby lubricity and fingerprint removability will further improve. On the other hand, when the molecular weight of the fluoropolyether chain is 20,000 or less, the obtainable surface layer will be more excellent in abrasion resistance.

### (Compound (A1))

The Compound (A1) has a structure represented by the following formula (A1).

[R^{f1}—(OR^{f11})_{y1}—O—R¹]ⱼ-L¹-(R¹¹-T¹¹)ₓ₁ (A1)
Symbols in the formula (A1) are as defined above.

R^{f1} is a C₁₋₂₀ fluoroalkyl group. The fluoroalkyl group may be linear, or may have a branch and/or a cyclic structure. In view of abrasion resistance, it is preferably a linear fluoroalkyl group, and in view of easiness in synthesis, etc., the number of carbon atoms in the fluoroalkyl group is preferably 1 to 6.

R^{f11} is a C₁₋₆ fluoroalkylene group, and when there is a plurality of R^{f11}, the plurality of R^{f11} may be the same or different from each other. (OR^{f11})_{y1} represents a fluoropolyether chain, y1 is an integer of 1 or more and is preferably 1 to 200.

(OR^{f11})_{y1} preferably has a structure represented by the following formula (G11).
—[(OG^{f1})ₘ₁(OG^{f2})ₘ₂(OG^{f3})ₘ₃(OG^{f4})ₘ₄(OG^{f5})ₘ₅(OG^{f6})ₘ₆]-(G11)
wherein
G^{f1} is a fluoroalkylene group having one carbon atom,
G^{f2} is a fluoroalkylene group having two carbon atoms,
G^{f3} is a fluoroalkylene group having three carbon atoms,
G^{f4} is a fluoroalkylene group having four carbon atoms,
G^{f5} is a fluoroalkylene group having five carbon atoms,
G^{f6} is a fluoroalkylene group having six carbon atoms,
m1, m2, m3, m4, m5 and m6 are each independently an integer of 0 or 1 or more,
and m1+m2+m3+m4+m5+m6 is preferably an integer of 1 to 200.

In the formula (G11), the order of binding of (OG^{f1}) to (OG^{f6}) is optional. m1 to m6 in the formula (G11) respectively means the numbers of (OG^{f1}) to (OG^{f6}), not the arrangement. For example, (OG^{f5})ₘ₅ means that the number of (OG^{f5}) is m5, not a block arranged structure of (OG^{f5})ₘ₅. Likewise, the order of description of (OG^{f1}) to (OG^{f6}) does not represent the order of binding of the respective units.

The fluoroalkylene group having 3 to 6 carbon atoms may be a linear fluoroalkylene group, or may be a fluoroalkylene group having a branch or a cyclic structure.

Specific examples of G^{f1} include —CF₂—, and —CHF—.

Specific examples of G^{f2} include —CF₂CF₂—, —CHFCF₂—, —CHFCHF—, —CH₂CF₂—, and 13 CH₂CHF—.

Specific examples of G^{f3} include —CF₂CF₂CF₂—, —CF₂CHFCF₂—, —CF₂CH₂CF₂—, —CHFCF₂CF₂—, —CHFCHFCF₂—, —CHFCHFCHF—, —CHFCH₂CF₂—, —CH₂CF₂CF₂—, —CH₂CHFCF₂—, —CH₂CH₂CF₂—, —CH₂CF₂CHF—, —CH₂CHFCHF—, —CH₂CH₂CHF—, —CF(CF₃)—CF₂—, —CF(CHF₂)—CF₂—, —CF(CH₂F)—CF₂—, —CF(CH₃)—CF₂—, —CF(CF₃)—CHF—, —CF(CHF₂)—CHF—, —CF(CH₂F)—CHF—, —CF(CH₃)—CHF—, —CF(CF₃)—CH₂—, —CF(CHF₂)—CH₂—, —CF(CH₂F)—CH₂—, —CF(CH₃)—CH₂—, —CH(CF₃)—CF₂—, —CH(CHF₂)—CF₂—, —CH(CH₂F)—CF₂—, —CH(CH₃)—CF₂—, —CH(CF₃)—CHF—, —CH(CHF₂)—CHF—, —CH(CH₂F)—CHF—, —CH(CH₃)—CHF—, —CH(CF₃)—CH₂—, —CH(CHF₂)—CH₂—, and —CH(CH₂F)—CH₂—.

Specific examples of G^{f4} include —CF₂CF₂CF₂CF₂—, —CHFCF₂CF₂CF₂—, —CH₂CF₂CF₂CF₂—, —CF₂CHFCF₂CF₂—, —CHFCHFCF₂CF₂—, —CH₂CHFCF₂CF₂—, —CF₂CH₂CF₂CF₂—, —CHFCH₂CF₂CF₂—, —CH₂CH₂CF₂CF₂—, —CHFCF₂CHFCF₂—, —CH₂CF₂CHFCF₂—, —CF₂CHFCHFCF₂—, —CHFCHFCHFCF₂—, —CH₂CHFCHFCF₂—, —CF₂CH₂CHFCF₂—, —CHFCH₂CHFCF₂—, —CH₂CH₂CHFCF₂—, —CF₂CH₂CH₂CF₂—, —CHFCH₂CH₂CF₂—, —CH₂CH₂CH₂CF₂—, —CHFCH₂CH₂CHF—, —CH₂CH₂CH₂CHF—, and —cycloC₄F₆—.

Specific examples of G^{f5} include —CF₂CF₂CF₂CF₂CF₂—, —CHFCF₂CF₂CF₂CF₂—, —CH₂CHFCF₂CF₂CF₂—, —CF₂CHFCF₂CF₂CF₂—, —CHFCHFCF₂CF₂CF₂—, —CF₂CH₂CF₂CF₂CF₂—, —CHFCHECF₂CF₂CF₂—, —CHECH₂CF₂CF₂CF₂—, —CF₂CF₂CHFCF₂CF₂—, —CHFCF₂CHFCF₂CF₂—, —CH₂CF₂CHFCF₂CF₂—, —CH₂CF₂CF₂CF₂CH₂—, and —cycloC₅F₈—.

Specific examples of G^{f6} include —CF₂CF₂CF₂CF₂CF₂CF₂—, —CF₂CF₂CHFCHFCF₂CF₂—, —CHFCF₂CF₂CF₂CF₂CF₂—, —CHFCHFCHFCHFCHFCHF—, —CHFCF₂CF₂CF₂CF₂CH₂—, —CH₂CF₂CF₂CF₂CF₂CH₂—, and -cycloC₆F₁₀—.

-cycloC₄F₆— means a perfluorocyclobutanediyl group, and its specific examples include a perfluorocyclobutene-1,2-diyl group. cycloC₅F₈— means a perfluorocyclopentanediyl group and its specific examples include a perfluorocyclopentan-1,3-diyl group. -cycloC₆F₁₀— means a perfluorocyclohexanediyl group and its specific examples include a perfluorocyclohexan-1,4-diyl group.

(OR^{f11})_{y1} particularly preferably has a structure represented by any one of the following formulae (G2) to (G4), in view of more excellent water/oil repellency, abrasion resistance and fingerprint stain removability.
(OG^{f1})ₘ₁-(OG^{f2})ₘ₂ formula (G2)
(OG^{f2})ₘ₂-(OG^{f4})ₘ₄ formula (G3)
(OG^{f3})ₘ₃ formula (G4)

Symbols in the formulae (G2) to (G4) are as defined for the above formula (G11).

In the formulae (G2) and (G3), the orders of binding of (OG^{f1}) and (OG^{f2}), and (OG^{f2}) and (OG^{f4}), are respectively optional. For example, in the formula (G2), (OG^{f1}) and (OG^{f2}) may alternately be arranged, (OG^{f1}) and (OG^{f2}) may be arranged respectively in blocks, or may be randomly arranged.

In the formula (G2), m1 is preferably 1 to 30, more preferably 1 to 20. m2 is preferably 1 to 30, more preferably 1 to 20.

In the formula (G3), m2 is preferably 1 to 30, more preferably 1 to 20. m4 is preferably 1 to 30, more preferably 1 to 20.

In the formula (G4), m3 is preferably 1 to 30, more preferably 1 to 20.

In the fluoropolyether chain (OR^{f11})_{y1}, the proportion of fluorine atoms [{number of fluorine atoms/(number of fluorine atoms+number of hydrogen atoms)}×100 (%)] is, in view of excellent water/oil repellency and fingerprint removability, preferably 60% or more, more preferably 70% or more, further preferably 80% or more. The upper limit is for example 100%.

The molecular weight of the fluoropolyether chain (OR^{f11})_{y1} moiety is, in view of abrasion resistance, preferably 2,000 to 20,000, more preferably 2,500 to 15,000, further preferably 3,000 to 10,000.

R¹ is an alkylene group or a fluoroalkylene group. The alkylene group and fluoroalkylene group as R¹ may be linear or may have a branch and/or a cyclic structure. In view of easiness in synthesis, etc., preferred is an alkylene group or a fluoroalkylene group that is linear or that has a branch, more preferred is an alkylene group or a fluoroalkylene group that is linear or that has a methyl group or a fluoromethyl group as a branch. The number of carbon atoms in R¹ is preferably 1 to 30, more preferably 1 to 20, further preferably 1 to 10, particularly preferably 1 to 6. In a case where j is 1 and L¹ is a single bond, R¹ is bonded to R¹¹. In such a case, the carbon atom in R¹, bonded to R¹¹, is bonded to at least one fluorine atom or fluoroalkyl group.

j represents the number of [R^{f1}—(OR^{f11})_{y1}—O—R¹] in one molecule and is an integer of 1 or more, preferably 1 to 20, more preferably 1 to 10, further preferably 1 to 4.

R¹¹ is an alkylene group in which the atom bonded to L¹ may be an etheric oxygen atom, and which may have an etheric oxygen atom between carbon atoms.

The alkylene group as R¹¹ may be linear, or may have a branch and/or a cyclic structure. It is preferably an alkylene group that is linear or that has a branch, more preferably a linear alkylene group, whereby the Compound (A1) will densely be arranged when forming a surface layer.

In a case where T¹¹ is —CH═CH₂, at least one R¹¹ has a carbon chain having 2 or more carbon atoms linked to each other, and in order that the first component will more efficiently be extracted, at least one R¹¹ preferably has a carbon chain having 4 or more carbon atoms linked to one another, more preferably has a carbon chain having 7 or more carbon atoms linked to one another, further preferably has a carbon chain having 10 or more carbon atoms linked to one another. The number of the linked carbon atoms in the carbon chain is, in view of easiness of production, preferably 28 or less, more preferably 18 or less. In a case where T¹¹ is —CH═CH₂, the carbon chain is linked to T¹¹.

On the other hand, in a case where T¹¹ is —SiR^{a11}_{z11}R^{a12}_{3-z11}, at least one R¹¹ has a carbon chain having 4 or more carbon atoms linked to one another, and in order that the first component will more efficiently be extracted, at least one R¹¹ preferably has a carbon chain having 6 or more carbon atoms linked to one another, more preferably has a carbon chain having 9 or more carbon atoms linked to one another, further preferably has a carbon chain having 12 or more carbon atoms linked to one another. The number of the linked carbon atoms in carbon chain is, in view of easiness of production, preferably 30 or less, more preferably 20 or less.

“Having a carbon chain having X or more carbon atoms linked to one another” means that R¹¹ has an alkylene group having X or more carbon atoms. In the case of an alkylene group having a branch and/or a cyclic structure, carbon atoms in the branch and the cyclic structure are included. Specifically, for example, —CH₂CH₂CH(—CH₂CH₃)—CH₂CH₂CH₂CH₂— has a carbon chain having 9 carbon atoms linked including carbon atoms in the branch.

R¹¹ is specifically represented by the following formula (g2).

*—(O)ₐ₁—(R^{g2}O)ₐ₂—R^{g2}—** (g2)
wherein R⁹² is an alkylene group having 1 or more carbon atoms, and the plurality of R^{g2} if any may be the same or different from each other,
a1 is 0 or 1,
a2 is an integer of 0 or more,
* is a binding site bonded to L¹, and
** is a binding site bonded to T¹¹,
provided that in a case where L¹ is a single bond, R¹¹ is directly bonded to R¹.

When a1 is 0, the atom having the binding site * is a carbon atom, and when a1 is 1, the atom having the binding site * is an oxygen atom. In the Compound (A1), a1 may be either 0 or 1 and is properly selected depending upon e.g. the synthesis.

a2 represents the number of repetition of R^{g2}O and in view of durability of the obtainable surface layer, is preferably 0 to 6, more preferably 0 to 3, further preferably 0 to 1.

The alkylene group as R^{g2} is the same as the alkylene group as R¹¹, and its preferred embodiment is also the same.

R¹¹ is further preferably a group represented by the following formula (g3), whereby the obtainable surface layer is more excellent in water/oil repellency and fingerprint stain removability and is also excellent in durability such as abrasion resistance.

*—(O)ₐ₁—R^{g3}—** (g3)
wherein R^{g3} is an alkylene group, and
a1, * and ** are as defined for the formula (g2).

The alkylene group as R^{g3} is the same as the alkylene group as R¹¹, and its preferred embodiment is also the same.

T¹¹ is —CH═CH₂, or a group represented by —SiR^{a11}_{z11}R^{a12}_{3-z11},
wherein R^{a11} is a hydroxy group or a hydrolysable group, and when there is a plurality of R^{a11}, the plurality of R^{a11} may be the same or different from each other,
R^{a12} is a non-hydrolysable group, and when there is a plurality of R^{a12}, the plurality of R^{a12} may be the same or different from each other, and
z11 is an integer of 1 to 3.

In a case where R^{a11} is a hydroxy group, it constitutes a silanol (Si—OH) group with the Si atom. The hydrolysable group is a group to be a hydroxy group (that is a silanol group) when hydrolyzed. The silanol group further undergoes intermolecular reaction to form a Si—O—Si bond. The silanol group undergoes a dehydration condensation reaction with a hydroxy group on the surface of a substrate (substrate-OH) to form a chemical bond (substrate-O—Si). The Compound (A1), which has 1 or more Ta11, impart excellent abrasion resistance to the formed surface layer.

The hydrolysable group as R^{a11} may, for example, be an alkoxy group, an aryloxy group, a halogen atom, an acyl group, an acyloxy group or an isocyanate group (—NCO). The alkoxy group is preferably a C₁₋₄ alkoxy group. The acyl group is preferably a C₁₋₆ acyl group. The acyloxy group is preferably a C₁₋₆ acyloxy group.

R^{a11} is, in view of easiness of production of the Compound (A1), preferably a C₁₋₄ alkoxy group or a halogen atom. The alkoxy group as R^{a11} is preferably a C₁₋₄ alkoxy group, whereby the Compound (A1) will be excellent in storage stability and outgassing at the time of reaction is suppressed, particularly preferably an ethoxy group in view of long-term storage stability, and particularly preferably a methoxy group in view of a short hydrolysis time. The halogen atom is preferably a chlorine atom.

The non-hydrolysable group as R^{a12} may, for example, be a hydrogen atom or a monovalent hydrocarbon group.

The hydrocarbon group may, for example, be an alkyl group, a cycloalkyl group, an alkenyl group or an allyl group, and the hydrocarbon group may be substituted by fluorine. In view of easiness of production, the hydrocarbon group is preferably an alkyl group. In view of easiness of production, the number of carbon atoms in the hydrocarbon group is preferably 1 to 6, more preferably 1 to 3, further preferably 1 to 2.

z11 is an integer of 1 to 3, and in view of adhesion to a substrate, preferably 2 or 3, more preferably 3.

In a case where T¹¹ is —SiR^{a11}_{z11}R^{a12}_{3-z11}, specific examples include —Si(OCH₃)₃, —SiCH₃(OCH₃)₂, —Si(OCH₂CH₃)₃, —SiCl₃, —Si(OCOCH₃)₃, —Si(NCO)₃, and —Si(OCH₂CF₃)₃. In view of handling efficiency in production, preferred is —Si(OCH₃)₃.

When there is a plurality of T¹¹ in one molecule, the plurality of T¹¹ may be the same or different from each other.

x1 represents the number of (R¹¹-T¹¹) in one molecule and is an integer of 1 or more, preferably 1 to 32, more preferably 1 to 18, further preferably 2 to 12.

L¹ is a single bond or a (j+x1)-valent group which may have N, O, S or Si and may have a branch point, and in which the atoms bonded to R¹ and R¹¹ are each independently N, O, S, Si, a carbon atom constituting the branch point, or a carbon atom having a hydroxy group or an oxo group (═O). The atom bonded to R¹ and the atom bonded to R¹¹ may be the same atom or may be different atoms.

In a case where L¹ is a single bond, R¹ and R¹¹ in the formula (A1) are directly bonded, and the Compound (A1) is represented by the following formula (A1′).

R^{f1}—(OR^{f11})_{y1}—O—R¹—R¹¹-T¹¹ (A1′)

Symbols in the formula (A1′) are as defined for the formula (A1).

In a case where L¹ is a trivalent or higher valent group, L¹ has at least one type of branch point (hereinafter referred to as “branch point P¹”) selected from the group consisting of C, N, Si, a cyclic structure and a (j+x1)-valent organopolysiloxane residue.

In a case where the branch point P¹ is N, the branch point P¹ is represented, for example, by *—N(—**)₂ or (*—)₂N—** . *is a binding site on the R¹ side, and ** is a binding site on the R¹¹ side.

In a case where the branch point P¹ is C, the branch point P¹ is represented, for example, by *—C(—*)₃, (*—)₂C(—**)₂, (*—)₃C—**, *—CR²⁹(—**)₂, or (*—)₂CR²⁹—**. * is a binding site on the R¹ side, ** is a binding site on the R¹¹ side, and R²⁹ is a monovalent group and may, for example, be a hydrogen atom, a hydroxy group, a hydrocarbon group, or an alkoxy group. The hydrocarbon group may be an aliphatic hydrocarbon group such as a linear alkyl group, an alkyl group having a branch or a cycloalkyl group, an aromatic hydrocarbon group such as a phenyl group, or a combination thereof.

In a case where the branch point P¹ is Si, the branch point P¹ is represented, for example, by *—Si(—**)₃, (*—)₂Si(—**)₂, (*—)₃Si—**, *—SiR²⁹(—**)₂, or (*—)₂SiR²⁹—**. * is a binding site on the R¹ side, ** is a binding site on the R¹¹ side, and R²⁹ is a monovalent group and may, for example, be a hydrogen atom, a hydroxy group, a hydrocarbon group, or an alkoxy group. The hydrocarbon group may be an aliphatic hydrocarbon group such as a linear alkyl group, an alkyl group having a branch or a cycloalkyl group, an aromatic hydrocarbon group such as a phenyl group, or a combination thereof.

The cyclic structure constituting the branch point P¹ is preferably at least one member selected from the group consisting of a 3- to 8-membered aliphatic ring, a 3- to 8-membered aromatic ring, a 3- to 8-membered heterocyclic ring and a condensed ring of 2 or more of such rings, more preferably a cyclic structure of any of the following formulae, in view of easy production of the Compound (A1) and whereby the obtainable surface layer will be more excellent in abrasion resistance, light resistance and chemical resistance. The cyclic structure may have a substituent such as a halogen atom, an alkyl group (which may have an etheric oxygen atom between carbon atoms), a cycloalkyl group, an alkenyl group, an allyl group, an alkoxy group or an oxo group (═O).

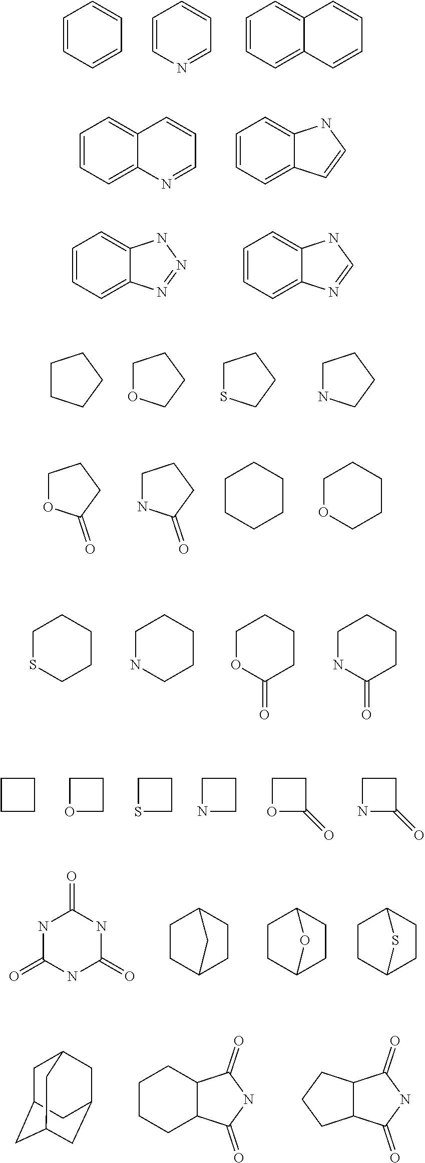

As the organopolysiloxane residue constituting the branch point P¹, the following groups may, for example, be mentioned. In the following formulae, R²⁵ is a hydrogen atom, an alkyl group, an alkoxy group or a phenyl group. The number of carbon atoms in the alkyl group or the alkoxy group as R²⁵ is preferably 1 to 10, more preferably 1. The plurality of R²⁵ may be the same or different from each other.

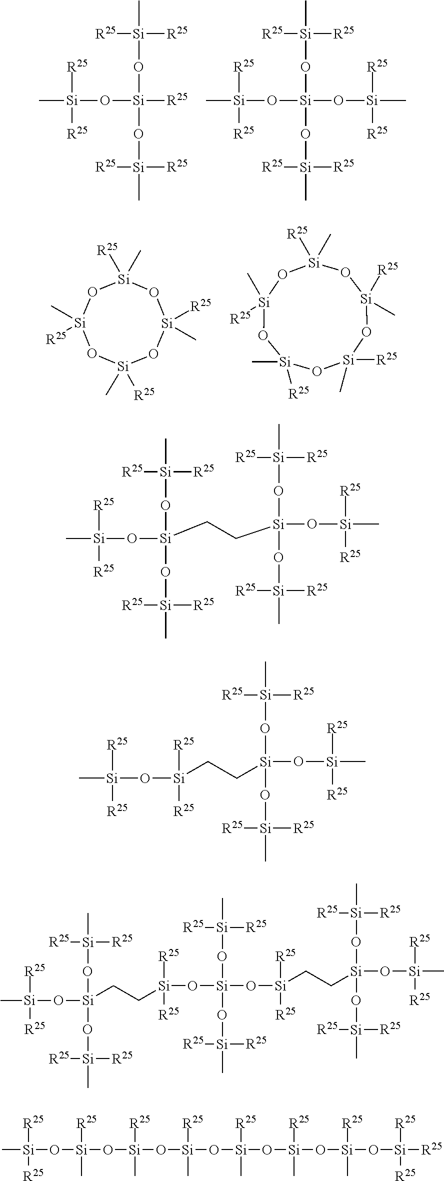

Bivalent or higher valent L¹ may have at least one bond selected from the group consisting of —C(O)N(R²⁶)—, —N(R²⁶)C(O)—, —C(O)O—, —OC(O)—, —C(O)—, —C(OH)—, —O—, —N(R²⁶)—, —S—, —OC(O)O—, —NHC(O)O—, —OC(O)NH—, —NHC(O)N(R²⁶)—, —SO₂N(R²⁶)—, —N(R²⁶)SO₂—, —Si(R²⁶)₂—, —OSi(R²⁶)₂—, —Si(CH₃)₂-Ph-Si(CH₃)₂— and a bivalent organopolysiloxane residue (hereinafter referred to as “bond B¹”).

In the above formulae, R²⁶ is a hydrogen atom, a C₁₋₆ alkyl group or a phenyl group, and Ph is a phenylene group. The number of carbon atoms in the alkyl group as R²⁶ is, in view of easy production of the Compound (A1), preferably 1 to 6, more preferably 1 to 3, further preferably 1 to 2.

As the bivalent organopolysiloxane residue, the following groups may, for example, be mentioned. In the following formulae, R²⁷ is a hydrogen atom, an alkyl group, an alkoxy group or a phenyl group. The number of carbon atoms in the alkyl group or the alkoxy group as R²⁷ is preferably 1 to 10, more preferably 1. The plurality of R²⁷ may be the same or different from each other.

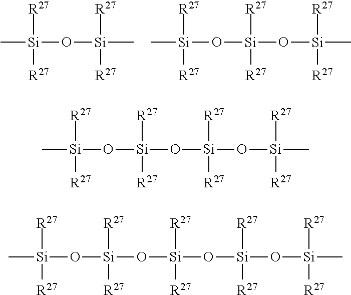

The bond B¹ is, in view of easy production of the Compound (A1), preferably at least one bond selected from the group consisting of —C(O)NR²⁶—, —N(R²⁶)C(O)—, —C(O)—, and —NR²⁶—, more preferably —C(O)NR²⁶—, —N(R²⁶)C(O)— or —C(O)—, whereby the obtainable surface layer will be more excellent in light resistance and chemical resistance.

In bivalent L¹, the atoms bonded to R¹ and R¹¹ are each independently N, O, S, Si, or a carbon atom having a hydroxy group or an oxo group (═O). That is, the atoms adjacent to R¹ and R¹¹ are respectively elements constituting the bond B¹. Specific examples of the bivalent L¹ include 1 or more of the bonds B¹ (for example *—B¹—**, *—B¹—R²⁸—B¹—**). R²⁸ is a single bond or a bivalent organic group, * is a binding site on the R¹ side, and ** is a binding site on the R¹¹ side.

In trivalent or higher valent L¹, the atoms bonded to R¹ and R¹¹ are each independently N, O, S, Si, a carbon atom constituting the branch point, or a carbon atom having a hydroxy group or an oxo group (═O). That is, the atoms adjacent to R¹ and R¹¹ are respectively elements constituting the bond B¹ or the branch point P¹. Specific examples of the trivalent or higher valent L¹ include 1 or more of the branch points P¹ (for example {(*—)ⱼP¹(—**)ₓ₁}, {(*—)ⱼP¹—R²⁸—P¹(—**)ₓ₁}), a combination of 1 or more of the branch points P¹ and 1 or more of the bonds B¹ (for example {*—B¹—R²⁸—P¹(—**)ₓ₁}, {*—B¹—R²⁸—P¹(—R²⁸—B¹—**)ₓ₁}). R²⁶ is a single bond or a bivalent organic group, * is a binding site on the R¹ side, and ** is a binding site on the R¹¹ side.

The bivalent organic group as R²⁸ may, for example, be a hydrocarbon group such as a bivalent aliphatic hydrocarbon group (e.g. an alkylene group or a cycloalkylene group), a bivalent aromatic hydrocarbon group (e.g. a phenylene group), and may have the bond B¹ between carbon atoms in the hydrocarbon group. The number of carbon atoms in the bivalent organic group is preferably 1 to 10, more preferably 1 to 6, further preferably 1 to 4.

The above L¹ is, in view of easy production of the Compound (A1), preferably a group represented by any one of the following formulae (L1) to (L7).

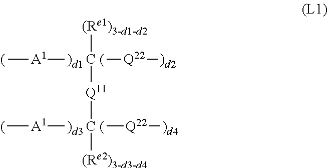

(-A¹-)_{d5}C(R^{e2})_{4-d5-d6}(-Q²²-)_{d6} (L2)
(-A²-)_{d7}N(-Q²³-)_{3-d7} (L3)
(-A³-)_{d8}Z¹(-Q²⁴-)_{d9} (L4)
(-A²-)_{d10}Si(R^{e3})_{4-d10-d11}(-Q²⁵-)_{d11} (L5)
-A¹-Q²⁶- (L6)
-A¹-CH(-Q²²-)-Si(R^{e3})_{3-d12}(-Q²⁵-)_{d12} (L7)

The group of each of the formulae (L1) to (L7) is linked to R¹ of the formula (A1) on the A¹, A² or A³ side, and is linked to R¹¹ on the Q²², Q²³, Q²⁴, Q²⁵ or Q²⁶ side.

A¹ is a single bond, —B³—, —B³—R³⁰—, or, —B³—R³⁰—B²—, wherein R³⁰ is an alkylene group or a group having —C(O)NR^{e6}—, —C(O)—, —CO(O)—, —NR^{e6}— or —O— between carbon atoms of an alkylene group having 2 or more carbon atoms, B² is —C(O)NR^{e6}—, —C(O)—, —NR^{e6}— or —O—, and B³ is —C(O)NR^{e6}—, —C(O)—, or —NR^{e6}—,
A² is a single bond or —B³—R³⁰—.
A³ is A¹ in a case where the atom in Z¹ to which A³ is bonded is a carbon atom, and is A² in a case where the atom in Z¹ to which A³ is bonded is a nitrogen atom,
Q¹¹ is a single bond, —O—, an alkylene group, or a group having —C(O)NR^{e6}—, —C(O)—, —NR^{e6}— or —O— between carbon atoms in an alkylene group having 2 or more carbon atoms,
Q²² is a single bond, —B³—, —R³⁰—B³— or —B²—R³⁰—B³—,
Q²³ is a single bond or —R³⁰—B³—,
Q²⁴ is Q²² in a case where the atom in Z¹ to which Q²⁴ is bonded is a carbon atom, and is Q²³ in a case where the atom in Z¹ to which Q²⁴ is bonded is a nitrogen atom,
Q²⁵ is a single bond or —R³⁰—B³—,
Q²⁶ is a single bond or —R³⁰—B³—,
Z¹ is a (d8+d9)-valent group having a cyclic structure, having a carbon atom or a nitrogen atom to which A³ is directly bonded and having a carbon atom or a nitrogen atom to which Q²⁴ is directly bonded,
R^{e1} is a hydrogen atom or a hydrocarbon group (e.g. an alkyl group or an aromatic hydrocarbon group such as a phenyl group),
R^{e2} is a hydrogen atom, a hydroxy group, a hydrocarbon group (e.g. an alkyl group or an aromatic hydrocarbon group such as a phenyl group) or an acyloxy group,
R^{e3} is a hydrocarbon group (e.g. an alkyl group or an aromatic hydrocarbon group such as a phenyl group),
R^{e6} is a hydrogen atom, a C16 alkyl group or a phenyl group,
d1 is an integer of 0 to 3, d2 is an integer of 0 to 3, and d1+d2 is an integer of 1 to 3,
d3 is an integer of 0 to 3, d4 is an integer of 0 to 3, and d3+d4 is an integer of 1 to 3,
d1+d3 is an integer of 1 to 5,
d2+d4 is an integer of 1 to 5,
d5 is an integer of 1 to 3, d6 is an integer of 1 to 3, and d5+d6 is an integer of 2 to 4,
d7 is 1 or 2,
d8 is an integer of 1 or more,
d9 is an integer of 1 or more,
d10 is an integer of 1 to 3, d11 is an integer of 1 to 3, and d10+d11 is an integer of 2 to 4, and
d12 is an integer of 1 to 3.

The directions of B² and B³ are not limited. When there is a plurality of A¹, the plurality of A¹ may be the same or different from each other. The same applies to A², A³, Q²², Q²³, Q²⁴, Q²⁵, R^{e1}, R^{e2}, and R^{e3}.

Further, d1+d3, d5, d7, d8, d10 are j, and d2+d4, d6, 3−d7, d9, d11, and 1+d12 are x1.

The number of carbon atoms in the alkylene group as R³⁰ is preferably 1 to 10, more preferably 1 to 6, further preferably 1 to 4, in view of easiness of production of the Compound (A¹), and whereby the obtainable surface layer will be more excellent in abrasion resistance, light resistance and chemical resistance. In a case where the alkylene group has a specific bond between carbon atoms, the lower limit of the number of carbon atoms in such an alkylene group is 2.

The cyclic structure in Z¹ may be the above described cyclic structure, and its preferred embodiment is also the same.

The number of carbon atoms in the alkyl group as R^{e1}, R^{e2} or R^{e3} is, in view of easy production of the Compound (A1), preferably 1 to 6, more preferably 1 to 3, further preferably 1 to 2.

The number of carbon atoms in the alkyl group moiety of the acyloxy group as R^{e2} is, in view of easiness of production of the Compound (A1), preferably 1 to 6, more preferably 1 to 3, further preferably 1 to 2.

d9 is preferably 2 to 6, more preferably 2 to 4, further preferably 2 or 3, in view of easiness of production of the Compound (A1), and whereby the obtainable surface layer will be more excellent in abrasion resistance and fingerprint stain removability.

As other embodiments of L¹, groups represented by the following formulae (L11) to (L17) may be mentioned.

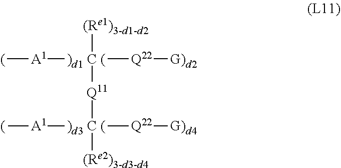

(-A¹-)_{d5}C(R^{e2})_{4-d5-d6}(-Q²²-G)_{d6} (L12)
(-A²-)_{d7}N(-Q²³-G)_{3-d7} (L13)
(-A³-)_{d8}Z¹(-Q²⁴-G)_{d9} (L14)
(-A²-)_{d10}Si(R^{e3})_{4-d10-d11}(-Q²⁵-G)_{d11} (L15)
-A¹-Q²⁶-G (L16)
-A¹-CH(-Q²²-G)-Si(R^{e3})_{3-d12}(-Q²⁵-G)_{d12} (L17)

The group of each of the formulae (L11) to (L17) is linked to R¹ of the formula (A1) on the A¹, A² or A³ side, and is linked to R¹¹ on the Q²², Q²³, Q²⁴, Q²⁵ or Q²⁶ side. G is the following group (G21), and 2 or more G which L¹ has may be the same or different from each other. Symbols other than G are the same as the symbols in the formulae (L1) to (L7).
—Si(R⁵¹)₃₋ₖ(-Q³-)ₖ (G21) The above group of the formula (G21) is linked to Q²², Q²³, Q²⁴, Q²⁵ or Q²⁶ on the Si side, and is linked to R¹¹ on the Q³ side. R⁵¹ is an alkyl group. Q³ is a single bond, or —R⁵²—B³—, R⁵² is an alkylene group, a group having —C(O)NR³²—, —C(O)—, —NR³²— or —O— between carbon atoms in an alkylene group having 2 or more carbon atoms, or —(OSi(R²²)₂)ₚ₁₁—O—, and 2 or more Q³ may be the same or different from each other. k is 2 or 3. R³² is a hydrogen atom, a C₁₋₆ alkyl group or a phenyl group. R²² is an alkyl group, a phenyl group or an alkoxy group, and the two R²² may be the same or different from each other. p11 is an integer of 0 to 5, and when p11 is 2 or more, the 2 or more (OSi(R²²)₂) may be the same or different from each other.

In a case where Q³ is —R⁵²—B³—, the number of carbon atoms in the alkylene group as R⁵² is preferably 1 to 10, more preferably 1 to 6, further preferably 1 to 4, in view of easy production of the Compound (A1), and whereby the obtainable surface layer will be more excellent abrasion resistance, light resistance and chemical resistance. In a case where the alkylene group has a specific bond between carbon atoms, the lower limit of the number of carbon atoms in such an alkylene group is 2.

The number of carbon atoms in the alkyl group as R⁵¹ is, in view of easy production of the Compound (A1), preferably 1 to 6, more preferably 1 to 3, further preferably 1 to 2.

The number of carbon atoms in the alkyl group as R²² is, in view of easy production of the Compound (A1), preferably 1 to 6, more preferably 1 to 3, further preferably 1 to 2.

The number of carbon atoms in the alkoxy group as R²² is, in view of excellent storage stability of the Compound (A1), preferably 1 to 6, more preferably 1 to 3, further preferably 1 to 2.

p11 is preferably 0 or 1.

The molecular weight of the group represented by L¹-(R¹¹-T¹¹)ₓ₁ is preferably 300 or more, more preferably 450 or more, further preferably 600 or more, whereby the obtainable surface layer will have more improved abrasion resistance.

On the other hand, the molecular weight is preferably 2,000 or less, more preferably 1,500 or less, further preferably 1,000 or less, in view of more excellent antifouling property.

The ratio of the molecular weight of the fluoropolyether chain represented by [R^{f1}—(OR^{f11})_{y1}—O—R¹]ⱼ to the molecular weight of the group represented by L¹-(R¹¹-T¹¹)ₓ₁, is preferably 10% or more, more preferably 12% or more, further preferably 13% or more, whereby the obtainable surface layer will have more improved abrasion resistance.

The upper limit of the above ratio is preferably 40% or less, more preferably 25% or less, in view of more excellent antifouling property.

As the Compound (A1), the following may, for example, be mentioned.

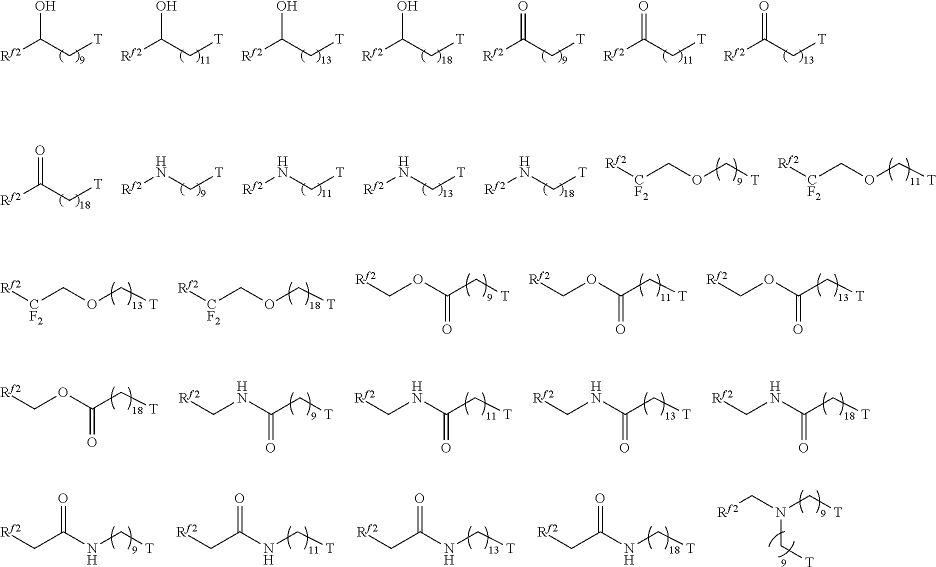

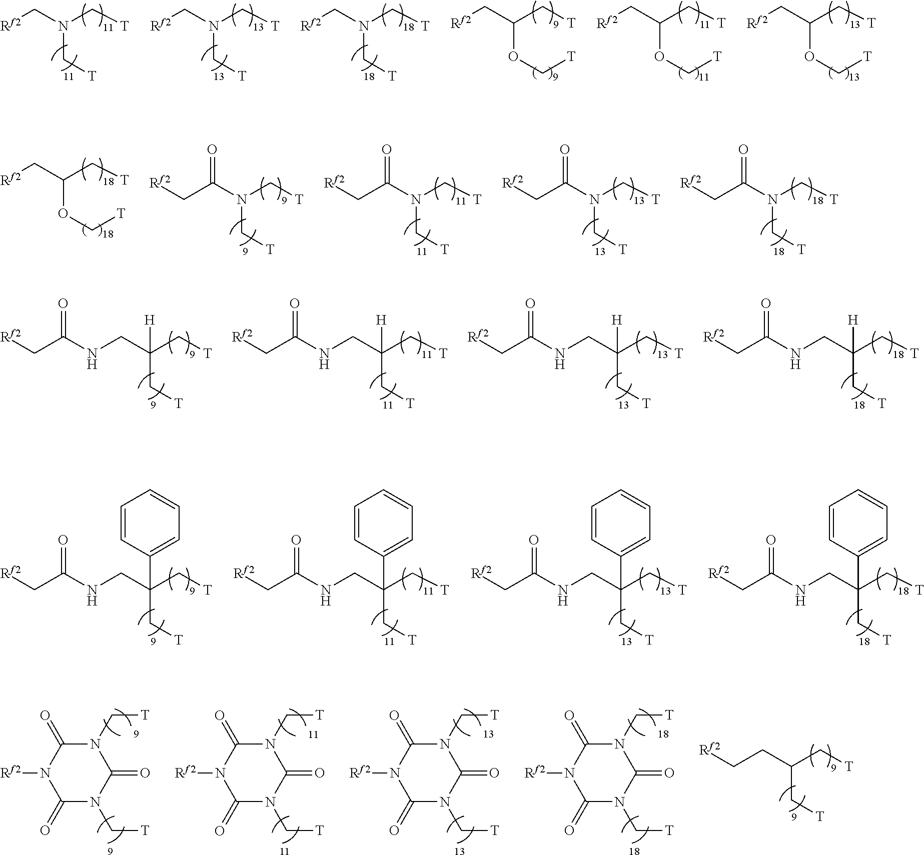

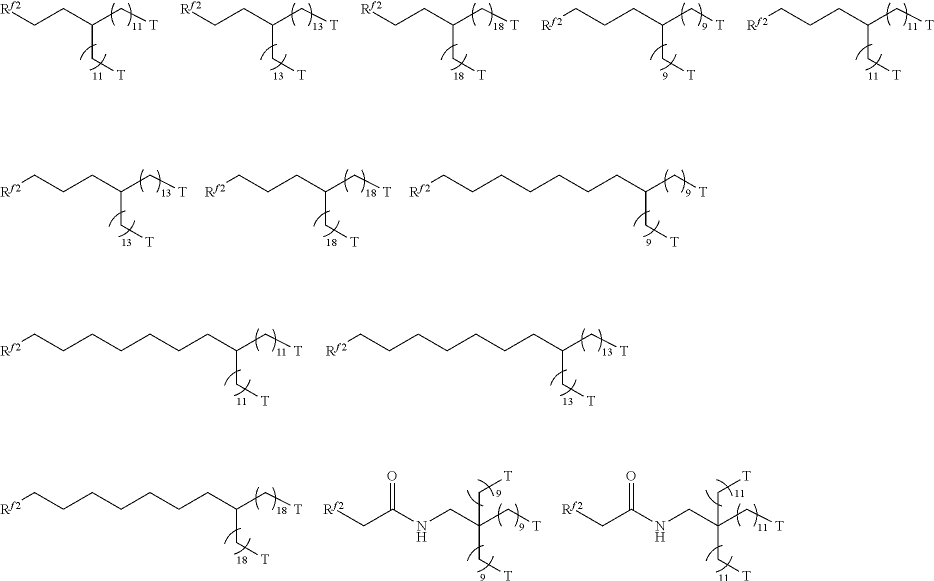

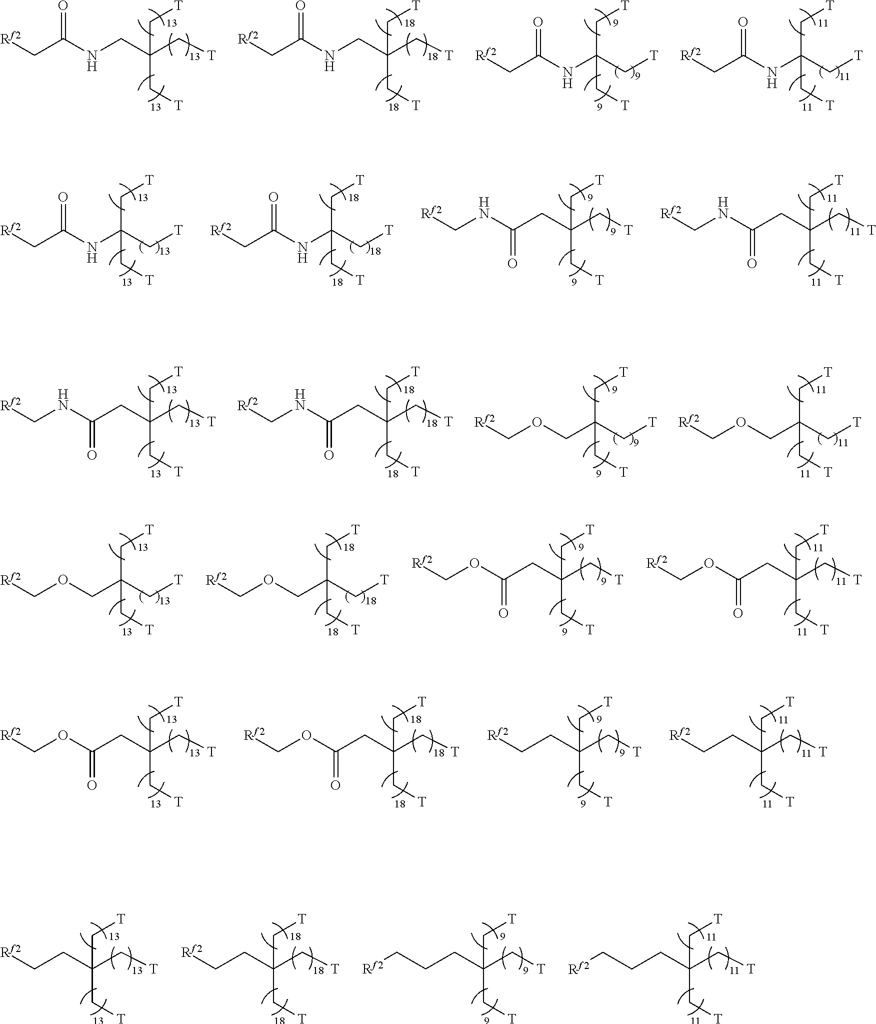

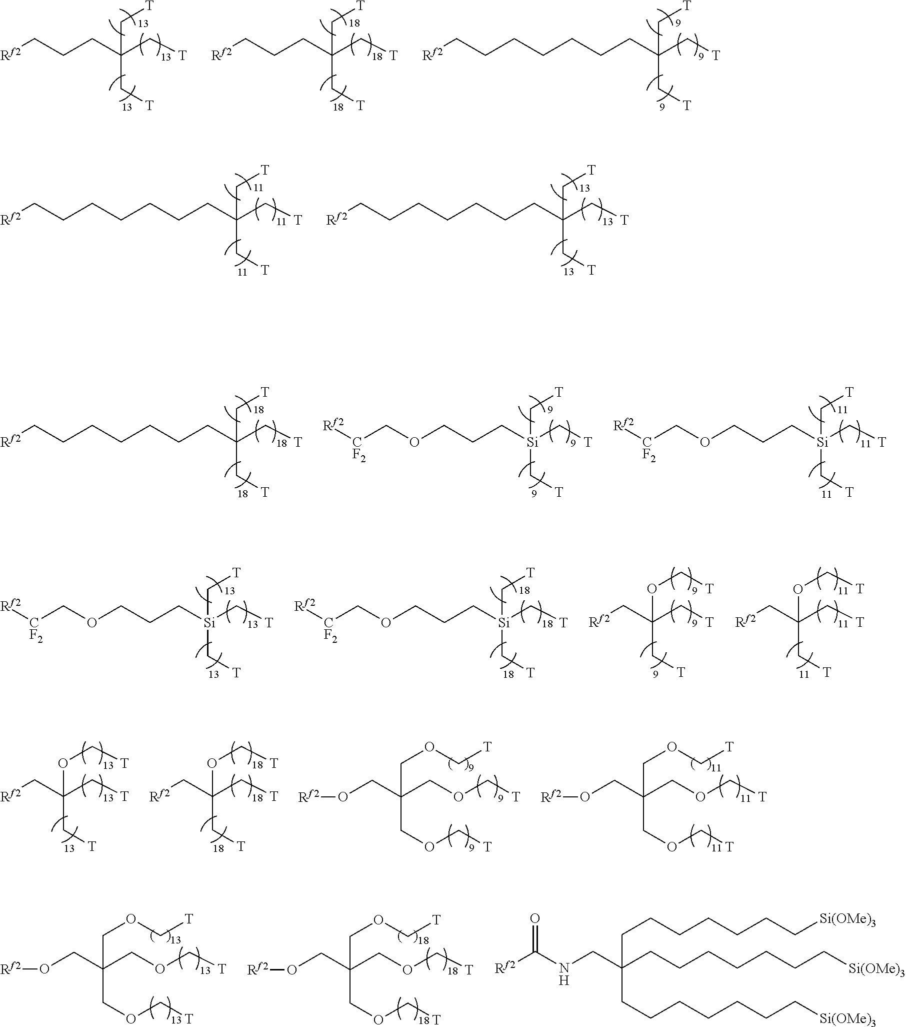

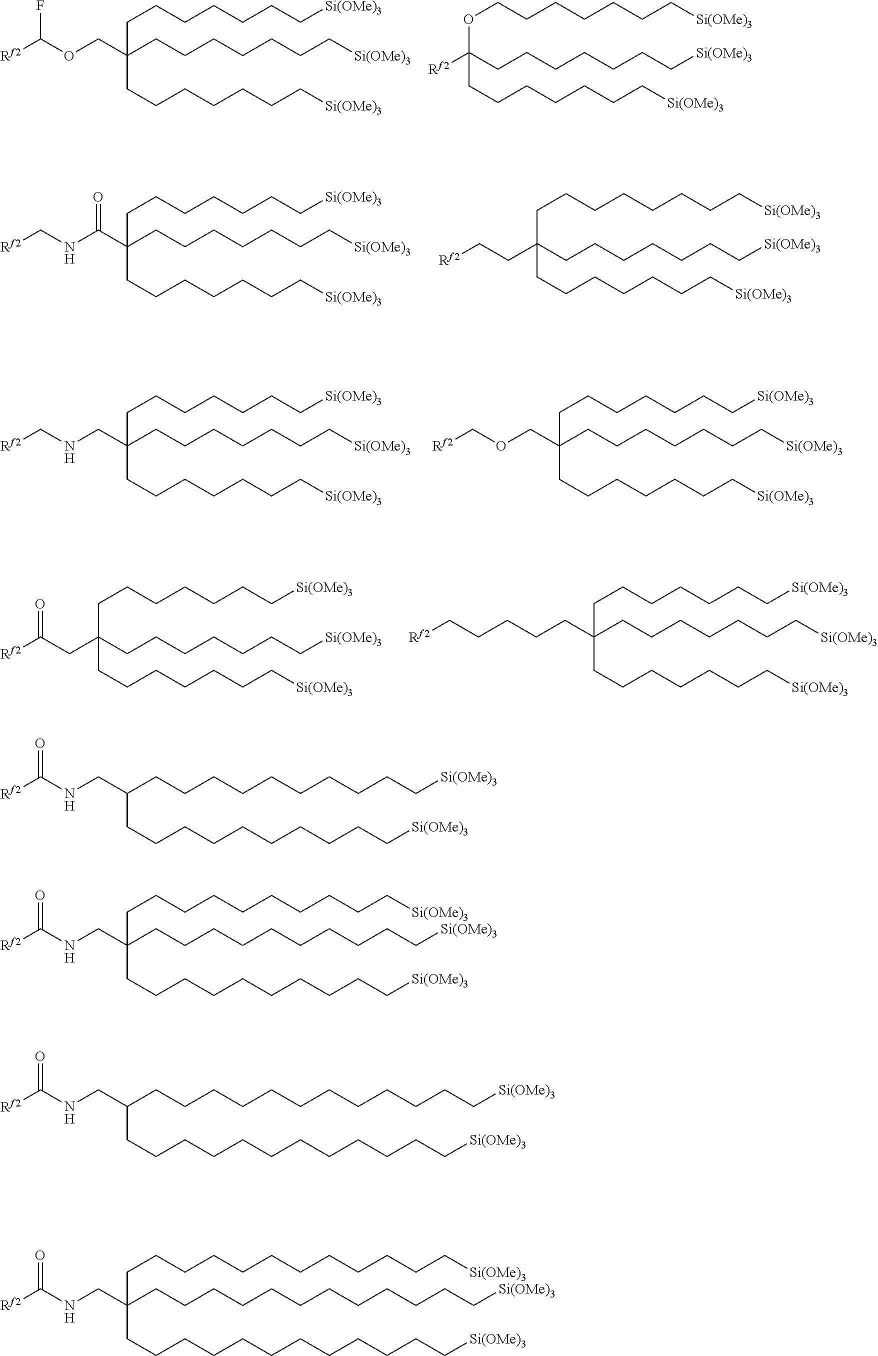

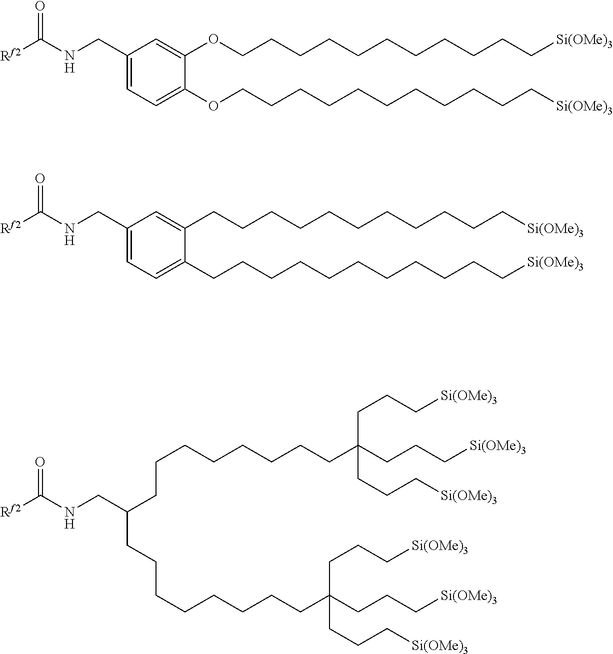

R^{f2} is each independently the same as [R^{f1}—(OR^{f11})_{y1}—O—R¹] or [R^{f1}—(OR^{f11})_{y1}—O—], T is the same as T¹¹, and Me represents a methyl group.

### (Compound (A2))

The Compound (A2) has a structure represented by the following formula (A2).

(T³¹-R³¹)ₓ₃-L³-R³—(OR^{f12})_{y2}—O—R²-L²-(R²¹-T²¹)ₓ₂ (A2)

Symbols in the formula (A2) are as defined above.

R^{f12} and (OR^{f12})_{y2} are the same as the above R^{f11} and (OR^{f11})_{y1}, and their preferred embodiments are also the same.

R² and R³ are each independently the same as R¹, and their preferred embodiments are also the same.

R²¹ and R³¹ are the same as R¹¹, and their preferred embodiments are also the same.

“Bonded to L¹” is read as “bonded to L²” in the case of R²¹, and is read as “bonded to L³” in the case of R³¹.

“Bonded to T¹¹” is read as “bonded to T²¹” in the case of R²¹, and is read as “bonded to T³¹” in the case of R³¹.

“Linked to T¹¹” is read as “linked to T²¹” in the case of R²¹, and is read as “linked to T³¹” in the case of R³¹.

“In the case of —SiR^{a12}_{z11}R^{a12}_{3-z11}” is read as “in the case of —SiR^{a21}_{z21}R^{a22}_{3-z21}”

In a case where L² is a single bond, R²¹ is directly bonded to R². In a case where L³ is a single bond, R³¹ is directly bonded to R³.

T²¹ and T³¹ are each independently —CH═CH₂, or —SiR^{a21}_{z21}R^{a22}_{3-z21}, R^{a21}, R^{a22} and z21 are each independently the same as R^{a1}, R^{a2} and z1 constituting T¹¹, and their preferred embodiments are also the same.

x2 and x3 are each independently the same as x1, and their preferred embodiments are also the same.

L² and L³ are each independently the same as L¹ wherein j is 1.

For example in a case where L² and L³ are a single bond, the Compound (A2) is represented by the following formula (A2′).

T³¹-R³¹—R³—(OR^{f12})_{y2}—O—R²—R²¹-T²¹ (A2′)

Symbols in the formula (A2′) are as defined for the formula (A2).

In a case where L² or L³ is a trivalent or higher valent group, the trivalent or higher valent L² or L³ has at least one type of branch point (hereinafter referred to as “branch point P²”) selected from the group consisting of C, N, Si, a cyclic structure and a (1+x2)-valent or (1+x3)-valent organopolysiloxane residue.

In a case where the branch point P² is N, the branch point P² is represented, for example, by *—N(—**)₂. * is a binding site on the R² or R³ side, and ** is a binding site on the R²¹ or R³¹ side.

In a case where the branch point P² is C, the branch point P² is represented, for example, by *—C(—**)₃, or *—CR²⁹(—**)₂. * is a binding site on the R² or R³ side, ** is a binding site on the R²¹ or R³¹ side, and R²⁹ is a monovalent group. R²⁹ may, for example, be a hydrogen atom, a hydroxy group, an alkyl group, or an alkoxy group.

In a case where the branch point P² is Si, the branch point P² is represented, for example, by *—Si(—**)₃, or *—SiR²⁹(—**)₂.

* is a binding site on the R² or R³ side, ** is a binding site on the R²¹ or R³¹ side, and R²⁹ is a monovalent group. R²⁹ may, for example, be a hydrogen atom, a hydroxy group, an alkyl group, or an alkoxy group.

The cyclic structure and the organopolysiloxane residue constituting the branch point P² are the same as for the branch point P¹, and their preferred embodiments are also the same.

The bivalent or higher valent L² and L³ may each independently have the bond B¹. The embodiment of the bond B¹ is as defined above, and its preferred embodiment is also the same.

In the bivalent L² or L³, the atoms bonded to R² and R²¹, or R³ and R³¹, are each independently N, O, S, Si, or a carbon atom having a hydroxy group or an oxo group (═O). That is, the atoms adjacent to R² and R²¹, or R³ and R³¹, are respectively elements constituting the bond B¹. Specific examples of the bivalent L² or L³ include 1 or more of the bonds B¹ (for example *—B¹—**, *—B¹—R²⁸—B¹—**). R²⁸ is a single bond or a bivalent organic group, * is a binding site on the R² or R³ side, and ** is a binding site on the R²¹ or R³¹ side.

In the trivalent or higher L² or L³, the atoms bonded to R² and R²¹, or R³ and R³¹, are each independently N, O, S, Si, a carbon atom constituting the branch point, or a carbon atom having a hydroxy group or an oxo group (═O). That is, the atoms adjacent to R² and R²¹, or R³ and R³¹, are respectively elements constituting the bond B¹ or the branch point P². Specific examples of the trivalent or higher valent L² or L³ include 1 or more of the branch points P² (for example {*—p²(—**)ₓ}, {*—P²—R²⁸—P²(—**)ₓ₁}), and a combination of 1 or more of the branch points P² and 1 or more of the bonds B¹ (for example {*—B¹—R²⁸—P²(—**)ₓ}, {*—B¹—R²⁸—P²(—R²⁸—B¹—**)ₓ}). x is x2 in the case of L², and is x3 in the case of L³. R²⁸ is a single bond or a bivalent organic group, * is a binding site on the R² or R³ side, and ** is a binding site on the R²¹ or R³¹ side.

The embodiment of the above R²⁸ is as described above, and its preferred embodiment is also the same.

L² or L³ are, in view of easy production of the Compound (A2), each independently preferably any one of groups represented by the following formulae (L21) to (L27).

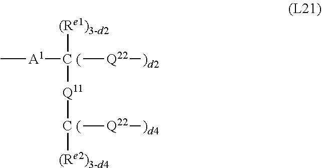

-A¹-C(R^{e2})_{4-d6}(-Q²²-)_{d6} (L22)
-A²-N(-Q²³-)₂ (L23)
-A³-Z¹(-Q²⁴-)_{d9} (L24)
-A²-Si(R^{e3})_{4-d11}(-Q²⁵-)_{d11} (L25)
-A¹-Q²⁶- (L26)
-A¹-CH(-Q²²-)-Si(R^{e3})_{3-d12}(-Q²⁵-)_{d12} (L27)

The group of each of the formulae (L21) to (L27) is linked to R² or R³ on the A¹, A² or A³ side, and is linked to R²¹ or R³¹ on the Q²², Q²³, Q²⁴, Q²⁵ or Q²⁶ side.

A¹, A², A³, Q¹¹, Q²², Q²³, Q²⁴, Q²⁵, Q²⁶, R^{e1}, R^{e2}R^{e3} and R^{e6} are as defined for L¹, and their preferred embodiments are also the same.

Z¹ is a (1+d9)-valent group having a cyclic structure, having a carbon atom or a nitrogen atom to which A³ is directly bonded and having a carbon atom or a nitrogen atom to which Q²⁴ is directly bonded,
d2 is an integer of 0 to 3, d4 is an integer of 0 to 3, and d2+d4 is an integer of 1 to 5,
d6 is an integer of 1 to 3,
d9 is an integer of 1 or more,
d11 is an integer of 1 to 3, and
d12 is an integer of 1 to 3.
d2+d4, d6, d9, d11, and 1+d12 are x2 or x3.

d9 is preferably 2 to 6, more preferably 2 to 4, further preferably 2 or 3, in view of easy production of the Compound (A2) and whereby the obtainable surface layer will be more excellent in abrasion resistance and fingerprint stain removability.

As other embodiments of L² and L³, groups represented by the following formulae (L31) to (L37) may be mentioned.

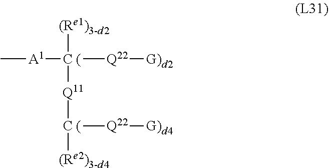

-A¹-C(R^{e2})_{4-d6}(-Q²²-G)_{d6} (L32)
-A²-N(-Q²³-G)₂ (L33)
A³-Z¹(-Q²⁴-G)_{d9} (L34)
-A²-Si(R^{e3})_{4-d11}(-Q²⁵-G)_{d11} (L35)
-A¹-Q²⁶-G(L36)
-A¹-CH(-Q²²-G)-Si(R^{e3})_{3-d12}(-Q²⁵-G)_{d12} (L37)

The group of each of the formulae (L31) to (L37) is bonded to R² or R³ on the A¹, A² or A³ side, and is bonded to R²¹ or R³¹ on the Q²², Q²³, Q²⁴, Q²⁵ or Q²⁶ side. G is the above group (G21), and its preferred embodiment is also the same. Symbols other than G are the same as the symbols in the formulae (L21) to (L27), and their preferred embodiments are also the same.

In the Compound (A2), the molecular weight of at least one of the group represented by L²-(R²¹-T²¹)ₓ₂ and the group represented by (T³¹-R³¹)ₓ₃-L³ is preferably 300 or more, more preferably 450 or more, further preferably 600 or more, whereby the obtainable surface layer will have more improved abrasion resistance.

On the other hand, the above molecular weight is preferably 2,000 or less, more preferably 1,500 or less, further preferably 1,000 or less, in view of more excellent antifouling property.

It is preferred that both the molecular weight of the group represented by L²-(R²¹-T²¹)ₓ₂ and the molecular weight of the group represented by (T³¹-R³¹)ₓ₃-L³ are within the above range.

The ratio of the molecular weight of the group represented by R³—(OR^{f12})_{y2}—O—R² to the sum of the molecular weight of the group represented by L²-(R²¹-T²¹)ₓ₂ and the molecular weight of the group represented by (T³¹-R³¹)ₓ₃-L³, is preferably 10% or more, more preferably 12% or more, further preferably 13% or more, whereby the obtainable surface layer will have more improved abrasion resistance.

The upper limit of the above ratio is, in view of more excellent antifouling property, preferably 40% or less, more preferably 25% or less.

As the Compound (A2), the following may, for example, be mentioned.

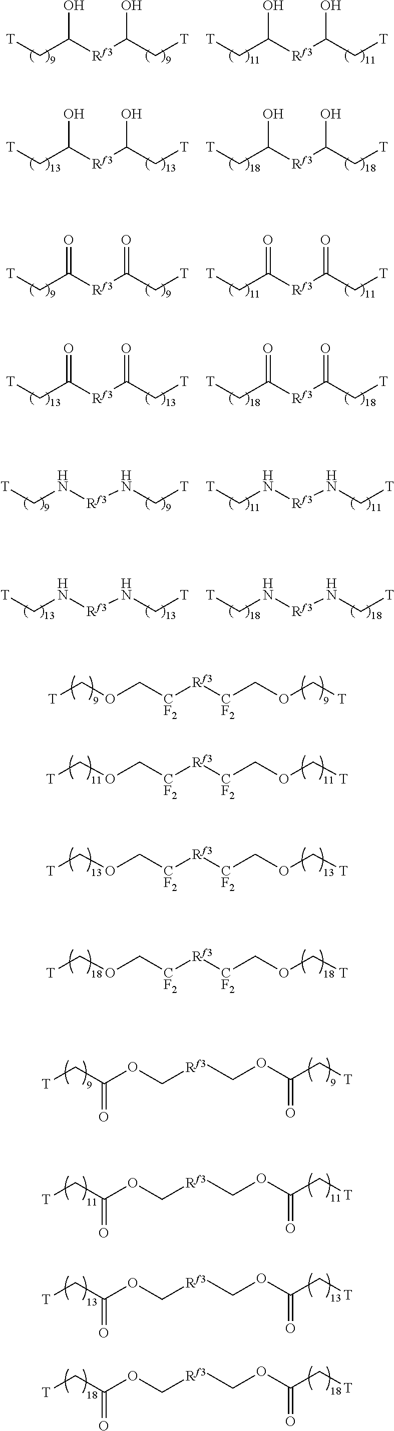

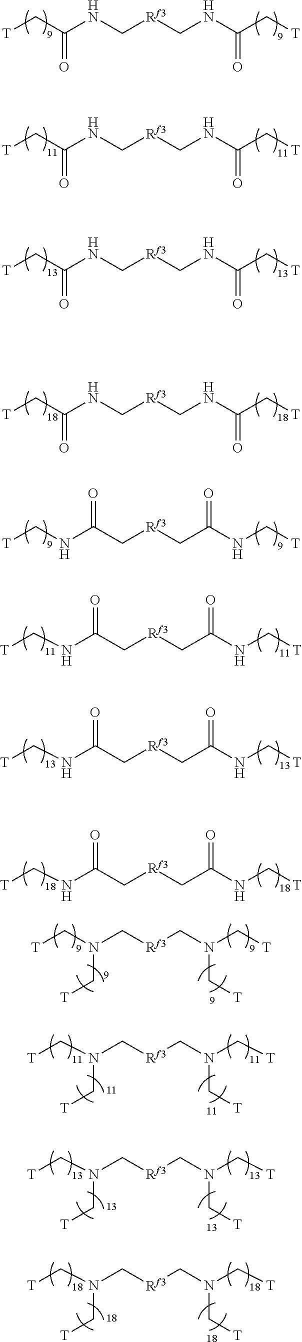

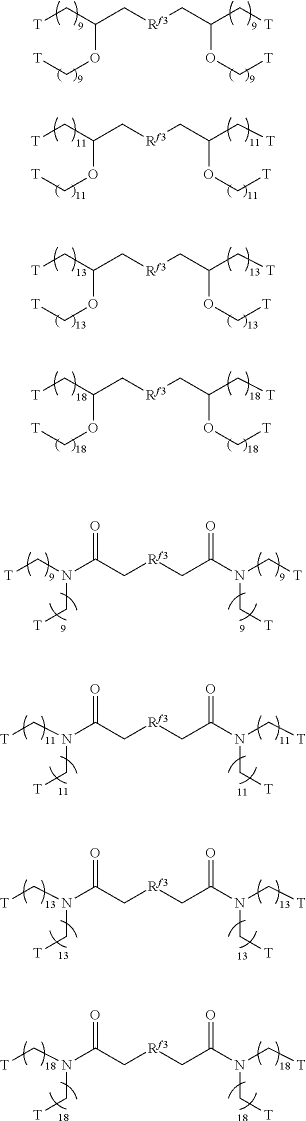

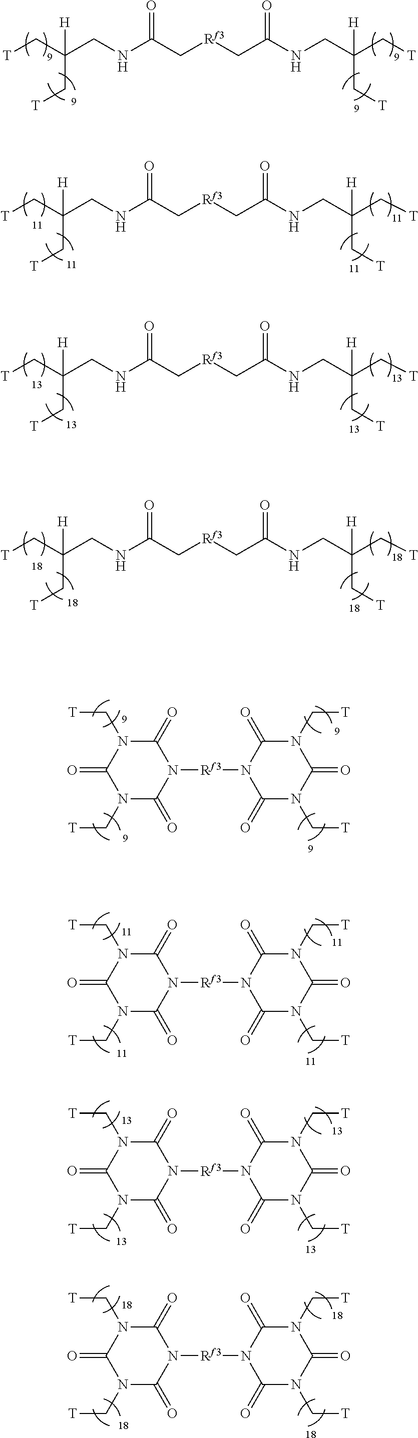

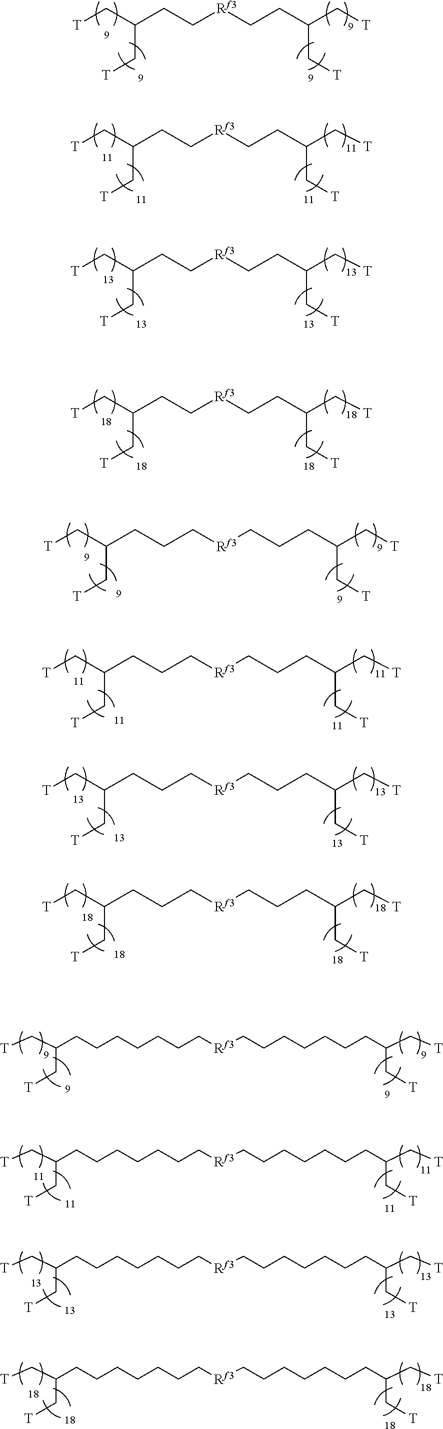

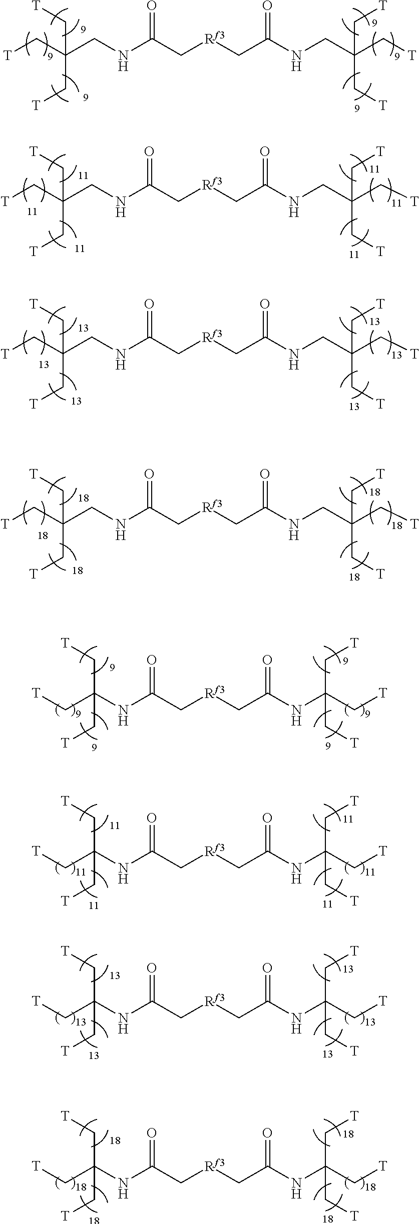

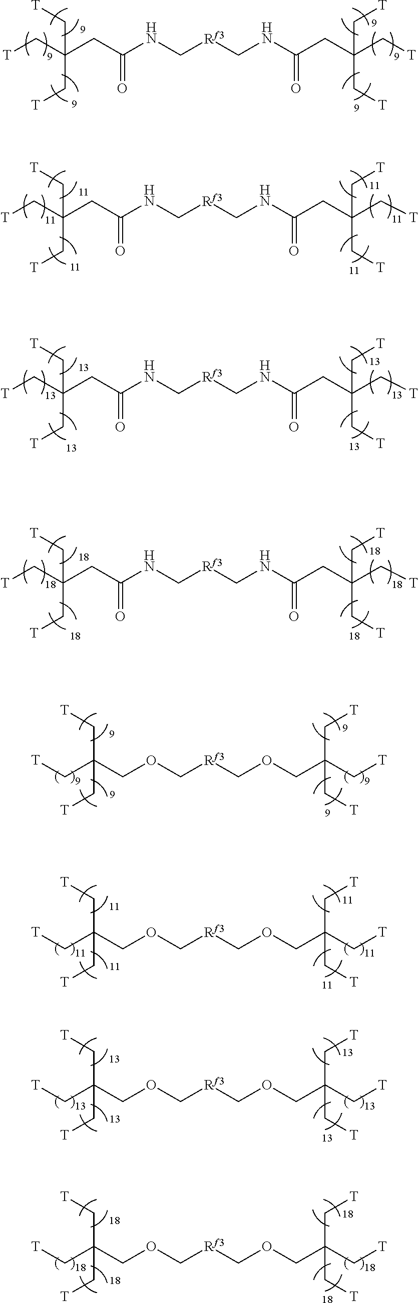

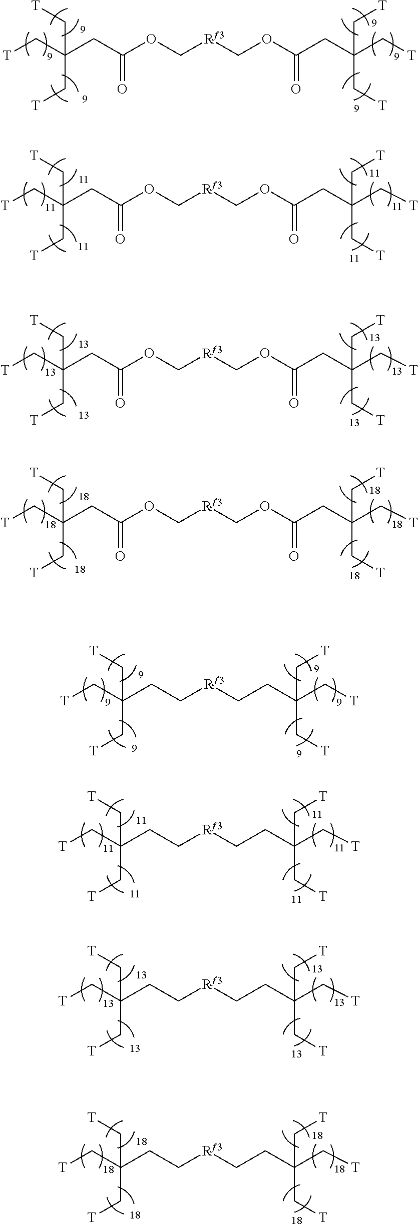

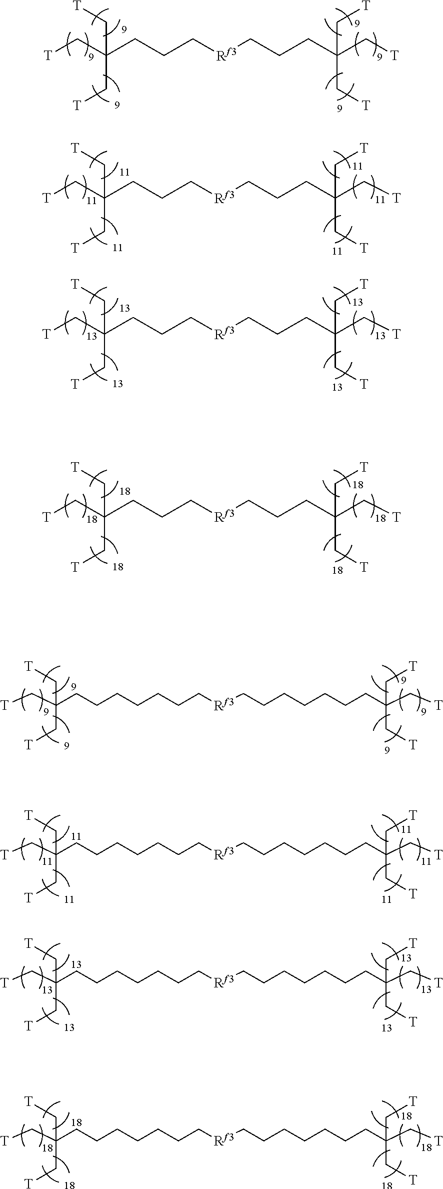

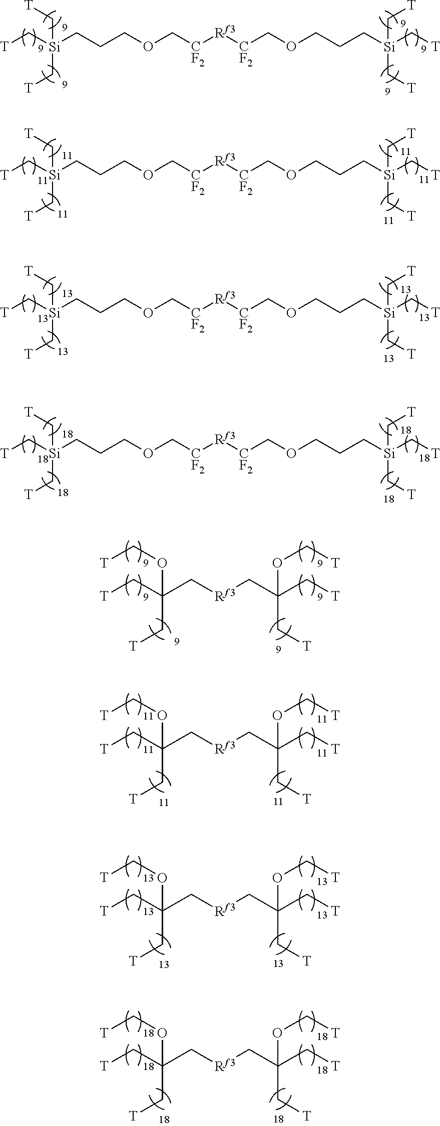

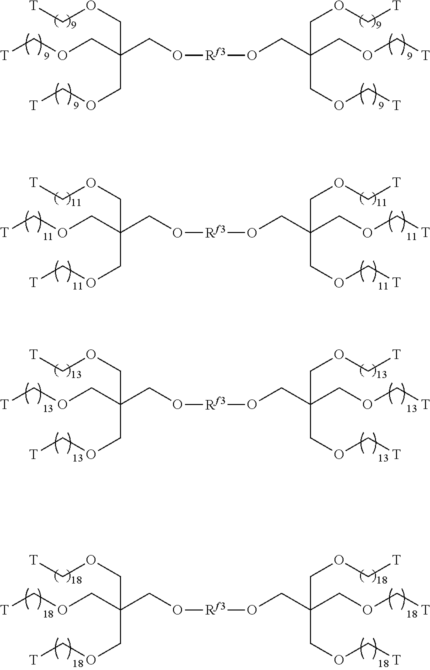

R^{f3} is each independently the same as [R³—(OR^{f12})_{y2}—O—R²] or [(OR^{f12})_{y2}—O], and T is the same as T²¹ or T³¹.

### (Compound (A3))

The Compound (A3) has a structure represented by the following formula (A3).

Q¹[-(OR^{f13})_{y3}—O—R⁴-L⁴-(R⁴¹-T⁴¹)ₓ₄]ᵣ₁ (A3)

Symbols in the formula (A3) are as defined above.

R^{f13} and (OR^{f13})_{y3} are the same as R^{f11} and (OR^{f11})_{y1}, and their preferred embodiments are also the same.

R⁴ is the same as R¹, and its preferred embodiment is also the same.

R⁴¹ is the same as R¹¹, and its preferred embodiment is also the same.

“Bonded to L¹” is read as “bonded to L⁴”.

“Bonded to T¹¹” is read as “bonded to T⁴¹”.

“Linked to T¹¹” is read as “linked to T⁴¹”.

“In the case of —SiR^{a11}_{z11}R^{a12}_{3-z11}” is read as “in the case of —SiR^{a41}_{z41}R^{a42}_{3-z41}”.

In a case where L⁴ is a single bond, R⁴¹ is directly bonded to R⁴.

T⁴¹ is —CH═CH₂ or —SiR^{a41}_{z41}R^{a42}_{3-z41}, R^{a41}, R^{a42} and z41 are each independently the same as R^{a1}, R^{a2} and z1 constituting T¹¹, and their preferred embodiments are also the same.

Q¹ is a r1-valent group having a branch point, and r1 is 3 or 4.

The branch point constituting Q¹ (hereinafter referred to as “branch point P³”) may be N, C, Si or a cyclic structure. Q¹ may have one branch point P³, or may have 2 or more branch points P³.

In a case where the branch point P³ is N, the branch point P³ is represented for example by N(—*)₃, or NR²⁹(—*)₂.

In a case where the branch point P³ is C, the branch point P³ may, for example, be C(—*)₄, CR²⁹(—*)₃, or C(R²⁹)₂(—*)₂.

In a case where the branch point P³ is Si, the branch point P³ may, for example, be Si(—*)₄, SiR²⁹(—*)₃, or Si(R²⁹)₂(—*)₂.

* is a binding site on the OR^{f13} side, and R²⁹ is a monovalent group. R²⁹ may, for example, be a hydrogen atom, a fluorine atom, a hydroxy group, an alkyl group, a fluoroalkyl group, or a fluoropolyether chain having no R⁴¹-T⁴¹.

The cyclic structure constituting the branch point P³ may be the same cyclic structure constituting the branch point P¹, and the cyclic structure may further have, as a substituent, in addition to the above substituent, at least one group selected from the group consisting of a fluorine atom, a fluoroalkyl group and a fluoropolyether chain having no R⁴¹-T⁴¹.

As Q¹, in view of easy production of the Compound (A3), preferred are groups represented by the following formulae (Q1) to (Q6).

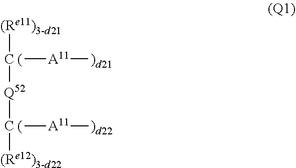

C(-A¹¹-)_{d23}(R^{e12})_{4-d23} (Q2)
N(-A¹²-)₃ (Q3)
Z¹(-A¹³-)_{d24} (Q4)
Si(-A¹²-)_{d25}(R^{e13})_{4-d25} (Q5)
CH(-A¹¹-)₂-Si(R^{e13})_{3-d26}(-A¹¹-)_{d26} (Q6)

In the formulae (Q1) to (Q6), A¹¹, A¹² or A¹³ is linked to (OR^{f13}).

A¹¹ is a single bond, —R⁴⁰—, or —B¹³—R⁴⁰—, R⁴⁰ is an alkylene group, a fluoroalkylene group or a group having —C(O)NR^{e16}—, —C(O)—, —CO(O)—, —NR^{e16}— or —O— between carbon atoms of an alkylene group or fluoroalkylene group having 2 or more carbon atoms, B¹³ is —C(O)NR^{e16}—, —C(O)—, —NR^{e16}— or —O—,
A¹² is a single bond or —R⁴⁰—,
A¹³ is A¹¹ in a case where the atom in Z¹ to which A¹³ is bonded is a carbon atom, and is A¹² in a case where the atom in Z¹ to which A¹³ is bonded is a nitrogen atom,
Z¹ is a r1-valent group having a cyclic structure having a carbon atom or nitrogen atom to which A¹³ is directly bonded,
Q⁵² is a single bond, —O—, an alkylene group, a fluoroalkylene group, or a group having —C(O)NR^{e16}—, —C(O)—, —NR^{e16}— or —O— between carbon atoms in an alkylene group or a fluoroalkylene group having 2 or more carbon atoms,
R^{e11} is a hydrogen atom, a fluorine atom, an alkyl group, a fluoroalkyl group, a fluoropolyether chain having no R⁴¹-T⁴¹, or a group having a repeating structure of -Q⁵²-C(R^{e11})_{3-d21}(-A¹¹-)_{d21} within a range where r1 is 3 to 4,
R^{e12} is a hydrogen atom, a fluorine atom, a hydroxy group, an alkyl group, a fluoroalkyl group, or a fluoropolyether chain having no R⁴¹-T⁴¹,
R^{e13} is an alkyl group or a fluoroalkyl group,
R^{e16} is a hydrogen atom, a C₁₋₆ alkyl group, a fluoroalkyl group or a phenyl group which may be substituted with fluorine,
d21 is an integer of 0 to 3, d22 is an integer of 0 to 3, and d21+d22 is an integer of 3 to 4,
d23 is 3 or 4,
d24 is 3 or 4,
d25 is 3 or 4, and
d26 is 1 or 2.

The direction of B¹³ is not limited. When there is a plurality of A¹¹, the plurality of A¹¹ may be the same or different from each other. The same applies to A¹², A¹³, R^{e11}, R^{e12} and R^{e13}.

The number of carbon atoms in the alkylene group or the fluoroalkylene group as R⁴⁰ is preferably 1 to 10, more preferably 1 to 6, further preferably 1 to 4, in view of easy production of the Compound (A3), and whereby the obtainable surface layer will be more excellent in abrasion resistance, light resistance and chemical resistance. In a case where the alkylene group has a specific bond between carbon atoms, the lower limit of the number of carbon atoms in such an alkylene group is 2.

The cyclic structure in Z¹ may be the cyclic structure constituting the branch point P3, and its preferred embodiment is also the same.

The number of carbon atoms in the alkylene group or the fluoroalkylene group as R^{e11}, R^{e12} or R^{e13} is, in view of easy production of the Compound (A3), preferably 1 to 6, more preferably 1 to 3, further preferably 1 to 2.

In the Compound (A3), the molecular weight of at least one of the groups represented by L⁴-(R⁴¹-T⁴)ₓ₄ is preferably 300 or more, more preferably 450 or more, further preferably 600 or more, whereby the obtainable surface layer will have more improved abrasion resistance.

On the other hand, the molecular weight is, in view of more excellent antifouling property, preferably 2,000 or less, more preferably 1,500 or less, further preferably 1,000 or less.

It is preferred that the molecular weights of all the groups represented by L⁴-(R⁴¹-T⁴¹)ₓ₄ are within the above range.

The ratio of the sum of the sum of the molecular weights of r1 groups represented by (OR^{f13})_{y3}—O—R⁴ and the molecular weight of the group represented by Q¹, to the sum of the molecular weights of r1 groups represented by L⁴-(R⁴¹-T⁴¹)ₓ₄, is preferably 10% or more, more preferably 12% or more, further preferably 13% or more, whereby the obtainable surface layer will have more improved abrasion resistance.

The upper limit of the above ratio is, in view of more excellent antifouling property, preferably 40% or less, more preferably 25% or less.

As the Compound (A3), the following may, for example, be mentioned.

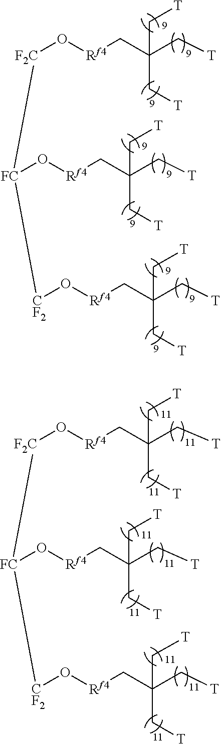

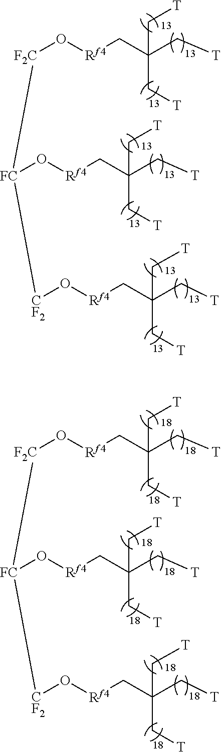

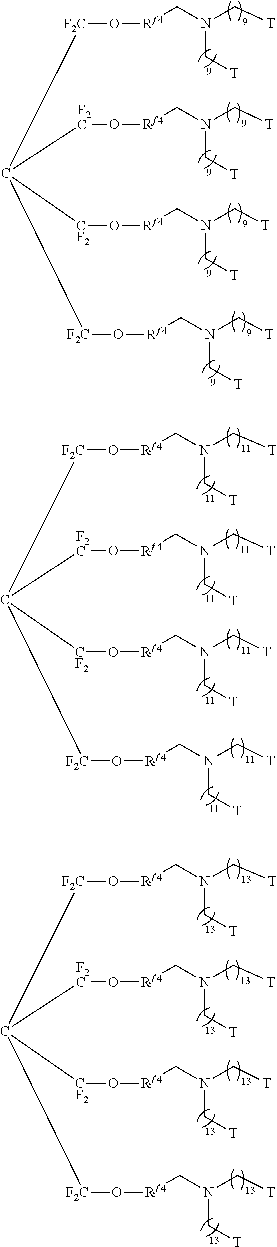

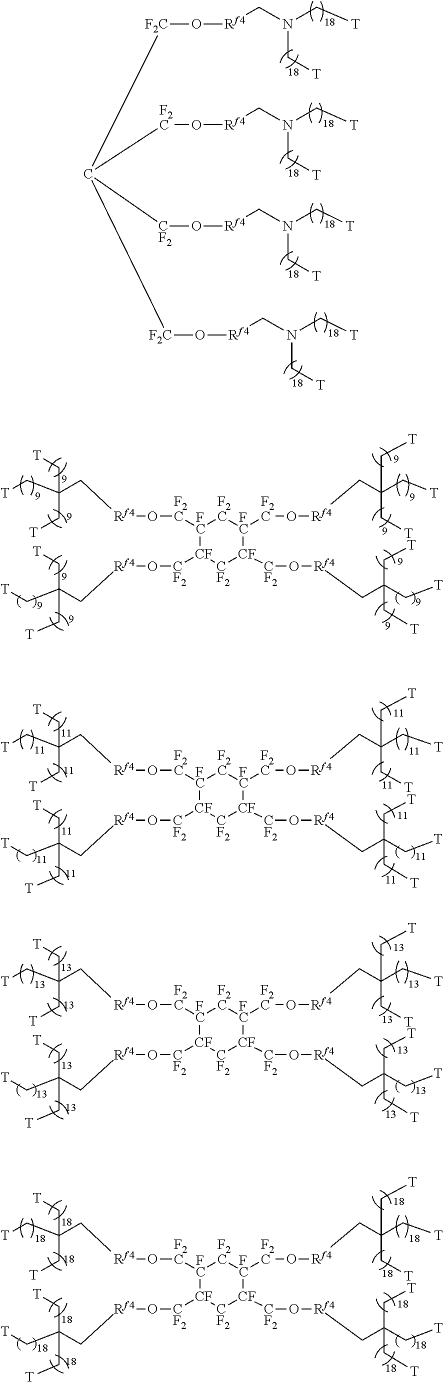

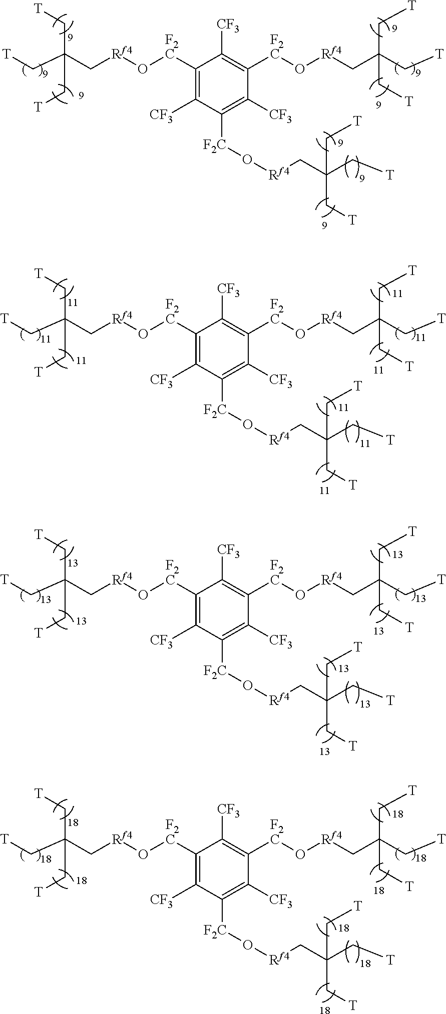

In the above compounds, {—O—R^{f4}—} is each independently the same as [—(OR^{f13})_{y3}—O—R⁴—], or [—(OR^{f13})_{y3}—O—], and T is the same as T⁴¹.

The present composition may contain a single type of the first component or may contain 2 or more types of the first components.

The content of the first component is, to the total mass of the present composition, preferably 1 to 99 mass %, more preferably 5 to 95 mass %, further preferably 10 to 90 mass %.

The concentration of the first component to the total molar amount of the first component and the second component contained in the present composition is preferably 5 to 95 mol %, more preferably 10 to 90 mol %.

### <Second Component>

The second component is at least one fluorinated ether compound selected from the group consisting of a compound represented by the following formula (B1) and a compound represented by the following formula (B2) and is a compound to be extracted in the present extraction method.
R^{f14}—(OR^{f15})_{y4}—O—R⁵—R^{b1} (B1)
R^{f19}—(OR^{f18})_{y6}—O—R⁷-L^{b1}-R⁶—O—(R^{f16}O)_{y5}—R^{f17} (B2)

In the formula (B1),
R^{f14} is a C₁₋₂₀ fluoroalkyl group,
R^{f15} is a C₁₋₆ fluoroalkylene group, and when there is a plurality of R^{f15}, the plurality of R^{f15} may be the same or different from each other,
R⁵ is an alkylene group which may have a substituent,
R^{b1} is a hydrogen atom, a chlorine atom, a bromine atom or an iodine atom, and
y4 is an integer of 1 or more.

R^{f17} and R^{f19} are each independently a C₁₋₂₀ fluoroalkyl group,
R^{f16} and R^{f18} are each independently a C₁₋₆ fluoroalkylene group, when there is a plurality of R^{f16}, the plurality of R^{f16} may be the same or different from each other, and when there is a plurality of R^{f18}, the plurality of R^{f18} may be the same or different from each other,
R⁶ and R⁷ are each independently an alkylene group which may have a substituent,
L^{b1} is a single bond or a bivalent linking group (excluding (OR^{f18})_{y7} and (R^{f16}O)_{y8}, y7 and y8 are each independently an integer of 1 or more), and
y5 and y6 are each independently an integer of 1 or more.

### (Compound (B1)

The Compound (B1) has a structure represented by the following formula (B1).

R^{f14}—(OR^{f15})_{y4}—O—R⁵—R^{b1} (B1)

Symbols in the formula (B1) are as defined above.

R^{f14} is a C₁₋₂₀ fluoroalkyl group.

The number of carbon atoms in the fluoroalkyl group is preferably 1 to 10, more preferably 1 to 6, particularly preferably 1 to 3, whereby the obtainable surface layer will be more excellent in water repellency.

The fluoroalkyl group may be any of linear, branched or cyclic.

The fluoroalkyl group is preferably a group having all hydrogen atoms in a fluoroalkyl group replaced with fluorine atoms (perfluoroalkyl group).

R^{f15} is a C₁₋₆ fluoroalkylene group.

The preferred embodiment of R^{f15} is the same as R^{f11} in the formula (A1). The preferred embodiment of (OR^{f15}) is the same as (OR^{f11}) the above formula (A1).

The number y4 of repetition of (OR^{f15}) is an integer of 1 or more. The preferred embodiment of y4 is the same as the number y1 of repetition of the above (OR^{f11}).

R⁵ is an alkylene group which may have a substituent.

The number of carbon atoms in the alkylene group which may have a substituent is preferably 1 to 30, more preferably 1 to 20, further preferably 1 to 10, particularly preferably 1 to 6. In a case where the substituent has a carbon atom, the number of the carbon atom in the substituent is not included in the number of carbon atoms in the alkylene group which may have a substituent.

The alkylene group which may have a substituent may be any of linear, branched and cyclic.

The substituent which the alkylene group may have may, for example, be a halogen atom, a hydroxy group or an amino group. The halogen atom may, for example, be a fluorine atom, a chlorine atom, a bromine atom or an iodine atom and is preferably a fluorine atom. In a case where the alkylene group has a fluorine atom, the alkylene group, that is a fluoroalkylene group, may be a group having all hydrogen atoms in the fluoroalkylene group replaced with fluorine atoms (perfluoroalkylene group).

R⁵ is, in view of more excellent extraction efficiency of the first component, preferably a fluoroalkylene group, more preferably a fluoroalkylene group having a hydrogen atom (that is a fluoroalkylene group excluding a perfluoroalkylene group), further preferably —R^{fb1}—R^{b11}. R^{fb1} is a C₁₋₆ perfluoroalkylene group, R^{b11} is a non-substituted C₁₋₆ alkylene group, and R^{b11} is bonded to R^{b1}. The sum of the number of carbons in R^{fb1} and the number of carbon atoms in R^{b11} is the same as the above number of carbon atoms in R⁵.

R^{b1} is a hydrogen atom, a chlorine atom, a bromine atom or an iodine atom.

R^{b1} is preferably a hydrogen atom or an iodine atom, more preferably a hydrogen atom.

The Compound (B1) may be used in combination of 2 or more.

Specific examples of the Compound (B1) include the following. PFPE in the following compounds is the same as R^{f14}—(OR^{f15})_{y4}— in the formula (B1), and its preferred embodiment is also the same. R^{fb1} is a C₁₋₆ perfluoroalkylene group, and represents the partial structure of R⁵ in the formula (B1). X in the following compounds is the same as R^{b1} in the formula (B1), and its preferred embodiment is also the same. R in the following compound is the same as the substituent which the alkylene group which may have a substituent represented by R⁵ in the formula (B1) may have, and its preferred embodiment is also the same.

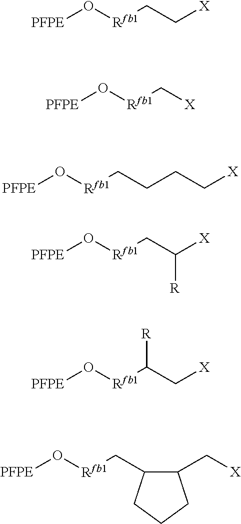

The method for producing the Compound (B1) is not particularly limited and description in WO2013/121984 may be mentioned.

### (Compound (B2))

The Compound (B2) has a structure represented by the following formula (B2).

R^{f19}—(OR^{f18})_{y6}—O—R⁷-L^{b1}-R⁶—O—(R^{f16}O)_{y5}—R^{f17} (B2)

Symbols in the formula (B2) are as defined above.

R^{f17} and R^{f19} are each independently a C₁₋₂₀ fluoroalkyl group. The preferred embodiments of R^{f17} and R^{f19} are the same as R^{f14} in the above formula (B1).

R^{f16} and R^{f18} are each independently a C₁₋₆ fluoroalkylene group. The preferred embodiments of R^{f16} and R^{f18} are the same as R^{f15} in the above formula (B1).

The number y5 of repetition of (R^{f16}O) is an integer of 1 or more. The preferred embodiment of y5 is the same as the number y4 of repetition of the above (OR^{f15}).

The number y6 of repetition of (OR^{f18}) is an integer of 1 or more. The preferred embodiment of y6 is the same as the number y4 of repetition of the above (OR^{f15}).

R⁶ and R⁷ are an alkylene group which may have a substituent. The preferred embodiments of R⁶ and R⁷ are the same as R⁵ in the above formula (B1).

The total number of carbon atoms in the alkylene group which may have a substituent as R⁶ and the alkylene group which may have a substituent as R⁷ is preferably 2 to 40, more preferably 2 to 20, further preferably 2 to 16. In a case where the substituent has a carbon atom, the number of the carbon atom in the substituent is not included in the number of carbon atoms in the alkylene group which may have a substituent.

R⁶ and R⁷ may be the same group or may be different groups, and are preferably the same group.

R⁶ is, in view of more excellent extraction efficiency of the first component, preferably a fluoroalkylene group, more preferably a fluoroalkylene group having a hydrogen atom (that is a fluoroalkylene group excluding a perfluoroalkylene group), further preferably —R^{fb2}—R^{b12}—. R^{fb2} is a C₁₋₆ perfluoroalkylene group, R^{b12} is a non-substituted C₁₋₆ alkylene group, and R^{b12} is bonded to L^{b1} (R⁶ in a case where L^{b1} is a single bond). The sum of the number of carbon atoms in Rfb2 and the number of carbon atoms in R^{b12} is the same as the number of carbon atoms in R⁶.

R⁷ is, in view of more excellent extraction efficiency of the first component, preferably a fluoroalkylene group, more preferably a fluoroalkylene group having a hydrogen atom (that is a fluoroalkylene group excluding a perfluoroalkylene group), further preferably —R^{b13}—R^{fb3}—. R^{fb3} is a C₁₋₆ perfluoroalkylene group, R^{b13} is a non-substituted C₁₋₆ alkylene group, and R^{b13} is bonded to L^{b1} (R⁷ in a case where L^{b1} is a single bond). The sum of the number of carbon atoms in R^{fb3} and the number of carbon atoms in R^{b13} is the same as the number of carbon atoms in R⁷.

L^{b1} is a single bond or a bivalent linking group (excluding (OR^{f18})_{y7} and (R^{f16}O)_{y8}, and y7 and y8 are each independently an integer of 1 or more).

Specific examples of the bivalent linking group include an alkylene group, an etheric oxygen atom, an amide bond and a group having these combined with each other. Among the bivalent linking groups, preferred are an alkylene group, a group having an alkylene group and an etheric oxygen atom combined, and a group having an alkylene group and an amide bond combined.

The bivalent linking group as L^{b1} does not include (OR^{f18})_{y7} and (R^{f16}O)_{y8}. The definitions of (OR^{f18}) and (R^{f16}O) are as defined above, and y7 and y8 are each independently an integer of 1 or more

L^{b1} is, in view of more excellent extraction efficiency of the first component, preferably a single bond.

The Compound (B2) may be used in combination of 2 or more.

Specific examples of the Compound (B2) include the following. PFPE in the following compounds is the same as R^{f19}—(OR^{f18})_{y6}— or —(R^{f16}O)_{y5}—R^{f17} in the formula (B2), and its preferred embodiment is also the same. R^{fb2} is a C₁₋₆ perfluoroalkylene group and represents the partial structure of R⁶ in the formula (B2). R^{fb3} is a C₁₋₆ perfluoroalkylene group and represents the partial structure of R⁷ in the formula (B2). R in the following compounds is the same as the substituent which the alkylene group which may have a substituent represented by R⁶ and R⁷ in the formula (B2) may have, and its preferred embodiment is also the same.

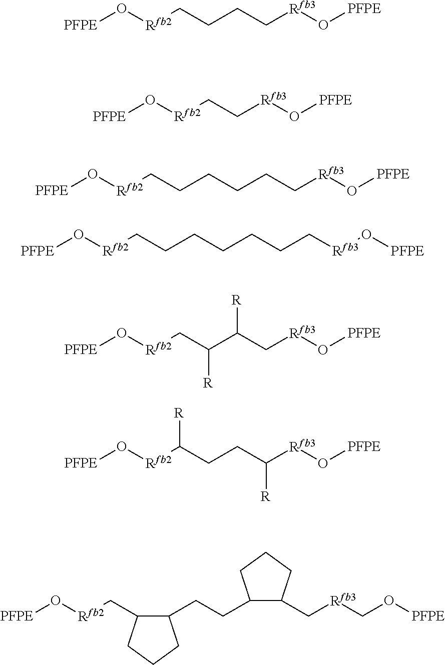

The method for producing the Compound (B2) is not particularly limited and may be a method of subjecting Compounds (B1) to a known coupling reaction.

The present composition may contain a single type of the second component or may contain 2 or more types of the second component.

The content of the second component is, to the total mass of the present composition, preferably 1 to 99 mass %, more preferably 5 to 95 mass %, further preferably 10 to 90 mass %.

### <Other Components>

The present composition may contain a component other than the first component and the second component (hereinafter sometimes referred to as “other component”).

Specific examples of such other component include an organic solvent containing a fluorine atom (hereinafter sometimes referred to as “fluorinated organic solvent”).

Specific examples of the fluorinated organic solvent include a fluorinated alkane, a fluorinated aromatic compound, a fluoroalkyl ether, a fluorinated alkylamine, a fluoroalcohol and a hydrofluoroolefin.

Specific examples of a preferred fluorinated alkane include C₄₋₈ compounds. Commercial products include C₆F₁₃H (manufactured by AGC Inc., ASAHIKLIN (registered trademark) AC-2000), C₆F₁₃C₂H₅ (manufactured by AGC Inc., ASAHIKLIN (registered trademark) AC-6000), and C₂F₅CHFCHFCF₃ (manufactured by Chemours, Vertrel (registered trademark) XF).

Specific examples of the fluorinated aromatic compound include hexafluorobenzene, trifluoromethylbenzene, perfuorotoluene and bis(trifluoromethyl)benzene.

The fluoroalkyl ether is preferably a C₄₋₁₂ compound. Commercial products include CF₃CH₂OCF₂CF₂H (manufactured by AGC Inc., ASAHIKLIN (registered trademark) AE-3000), C₄F₉OCH₃ (manufactured by 3M, Novec (registered trademark) 7100), C₄F₉OC₂H₅ (manufactured by 3M, Novec (registered trademark) 7200), and C₂F₅CF(OCH₃)C₃F₇ (manufactured by 3M, Novec (registered trademark) 7300).

Specific examples of the fluorinated alkylamine include perfluorotripropylamine and perfluorotributylamine.

Specific examples of the fluoroalcohol include 2,2,3,3-tetrafluoropropanol, 2,2,2-trifluoroethanol and hexafluoroisopropanol.

Specific examples of the hydrofluoroolefin include 1-chloro-2,3,3-trifluoro-1-propene (HCFO-1233yd), a reaction product of methanol with 1,1,1,2,2,3,4,5,5,6,6,7,7,7-tetradecafluoro-3-heptene, and a reaction product of methanol with 1,1,1,2,3,4,4,5,5,6,6,7,7,7-tetradecafluoro-3-heptene. Commercial products include AMOLEA (registered trademark) AS-300 manufactured by AGC Inc., and Opteon (registered trademark) (SF01, SF05, SF10, SF30, SF33, SF70, SF79, SF80) manufactured by Chemours.

In a case where the present composition contains the fluorinated organic solvent, the content of the fluorinated organic solvent is, to the total mass of the present composition, preferably 67 mass % or less, more preferably 50 mass % or less, further preferably 33 mass % or less.

### Non-Fluorinated Organic Solvent

The non-fluorinated organic solvent is used for extraction of the first component in an extraction step described later. Since the first component and the non-fluorinated organic solvent are well compatible, the first component can efficiently be extracted by using the non-fluorinated organic solvent.

The non-fluorinated organic solvent may be any organic solvent containing no fluorine atom, and is preferably a non-halogenated solvent in view of more excellent extraction efficiency of the first component.

The non-halogenated solvent is an organic solvent containing no halogen atom and may, for example, be a hydrocarbon-based organic solvent, an alcohol-based organic solvent, a ketone-based organic solvent, an ether-based organic solvent, an ester-based organic solvent, an amide-based organic solvent, or a sulfoxide-based organic solvent.

The hydrocarbon-based organic solvent is a compound composed solely of hydrogen atoms and carbon atoms and may be any of an aromatic hydrocarbon, an aliphatic hydrocarbon and an unsaturated hydrocarbon.

The aromatic hydrocarbon may, for example, be benzene, toluene or xylene.

The aliphatic hydrocarbon may, for example, be n-hexane, n-heptane, n-octane or n-decane.

The unsaturated hydrocarbon may, for example, be cyclopentene, hexene, heptene or butene.

The ketone-based organic solvent may, for example, be acetone, methyl ethyl ketone, methyl isobutyl ketone, 2-hexanone, cyclohexanone, methyl amino ketone, 2-heptanone, diisobutyl ketone or diacetone alcohol.

The alcohol-based organic solvent may, for example, be methanol, ethanol, propanol, isopropanol or butanol. A glycol-based solvent described later is not included in the alcohol-based solvent.

The ether-based organic solvent may, for example, be diethyl ether, diisopropyl ether, methyl t-butyl ether, dimethoxyethane, 1,4-dioxane, 1,3-dioxolane, tetrahydrofuran, cyclopentyl methyl ether, 4-methyltetrahydrofuran, 2-methyltetrahydrofuran, or a glycol-based solvent.

The glycol-based solvent may, for example, be a mono- or di-alkylene glycol mono- or di-alkyl ether, or a mono- or di-alkylene glycol mono- or di-alkyl ether acetate. The alkylene group is preferably an ethylene group or a propylene group. The alkyl group is preferably a C₁₋₄ alkyl group, more preferably a methyl group or an ethyl group. The glycol-based solvent may be more specifically ethylene glycol monomethyl ether, ethylene glycol monoethyl ether, propylene glycol monomethyl ether, propylene glycol monoethyl ether or propylene glycol monobutyl ether.

The ester-based organic solvent may, for example, be ethyl acetate, isopropyl acetate, butyl acetate, amyl acetate, methyl formate, methyl butyrate, butyl butyrate, methyl lactate, ethyl lactate, methyl 3-methoxypropionate, ethyl 3-methoxypropionate, methyl 2-hydroxyisobutyrate, ethyl 2-hydroxyisobutyrate, or an esterified compound of the glycol-based solvent and a carboxylic acid such as acetic acid.

The amide-based organic solvent may, for example, be N,N-dimethylformamide.

The sulfoxide-based organic solvent may, for example, be dimethyl sulfoxide.

In view of more excellent extraction efficiency of the first component, the relative permittivity of the non-halogenated solvent is preferably 30 or less, more preferably 20 or less, further preferably 10 or less.

In this specification, the relative permittivity of the organic solvent means the relative permittivity at 25° C., and is measured with a known liquid dielectric constant meter.

The non-halogenated solvent is, in view of more excellent extraction efficiency of the first component, preferably a hydrocarbon-based solvent.

The non-fluorinated organic solvent may be used singly or in combination of 2 or more

In the extraction step, the amount of the non-fluorinated organic solvent used is, per 100 parts by mass of the present composition, preferably 50 to 3,000 parts by mass, more preferably 100 to 2,000 parts by mass, further preferably 300 to 1,500 parts by mass.

When the amount of the non-fluorinated organic solvent used is 50 parts by mass or more, the amount of the first component that elutes in the non-fluorinated organic solvent increases, and the first component will more efficiently be extracted.

When the amount of the non-fluorinated organic solvent used is 3,000 parts by mass or less, the second component can efficiently be removed.

The mass ratio of the amount of the non-fluorinated organic solvent used to the amount of the first component used is, in view of more excellent extraction efficiency of the first component, preferably 50 to 15,000, more preferably 100 to 10,000, further preferably 300 to 7,500.

In a case where the present composition contains the fluorinated organic solvent, the mass ratio of the amount of the non-fluorinated organic solvent used to the amount of the fluorinated organic solvent used is, in view of more excellent extraction efficiency of the first component, preferably 1 or more, more preferably 3 or more, further preferably 5 or more.

### Extraction Step

The present extraction method includes a step of extracting the first component from the present composition using the non-fluorinated organic solvent (hereinafter sometimes referred to as “extraction step”). By this step, an eluate in which the first component more selectively elutes in the non-fluorinated organic solvent than the second component can be obtained.

As an example of a preferred embodiment of the extraction step, such a method may be mentioned that a mixture of the present composition and the non-fluorinated organic solvent is stirred and then left at rest to separate into an eluate in which the first component more selectively elutes in the non-fluorinated organic solvent than the second component, and components that have not eluted in the non-fluorinated organic solvent, and the layer of the eluate is recovered.

The temperature in the extraction step (for example, the temperature of the above mixture) is preferably 0 to 50° C., more preferably 15 to 35° C.

The extraction step may include removal treatment of removing the non-fluorinated organic solvent from the eluate.

A specific example of the removal treatment includes distillation (for example vacuum distillation). Distillation may be conducted e.g. by a known distillation apparatus.

The extraction step may include filtration treatment of removing solids that may be included in the above mixture or eluate. The filtration treatment may be conducted e.g. by a filtration filter.

The extraction step may be conducted only once, or may be conducted twice or more. In a case where the extraction step is conducted twice or more, an eluate containing the first component with higher purity can be obtained.

In the second or subsequent extraction step, it is preferred to use a component obtained by removing the non-fluorinated organic solvent and solids from the eluate obtained in the previous extraction step.

### EXAMPLES

Now, the present invention will be described in detail with reference to Examples. Examples 1 to 3 are Examples of the present invention, and Examples 4 to 6 are Comparative Examples. It should be understood that the present invention is by no means restricted to such specific Examples Amounts of the respective components in Tables described later are based on mass.

### Synthesis of Compound A1-1

### <Synthesis of Compound X1>

Into a reactor, 2.0 g of dimethyl malonate, 10.6 g of 11-bromo-1-undecene (CH₂═CH(CH₂)₉—Br), 8.4 g of potassium carbonate and 50.0 g of dimethylformamide (DMF) were put and stirred in a nitrogen atmosphere at 85° C. After completion of the reaction, hydrochloric acid was added, an organic phase was recovered, and the recovered organic phase was concentrated by an evaporator to obtain 6.1 g of Compound X1.

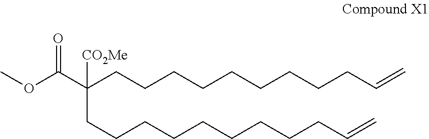

wherein Me is a methyl group.

### <Synthesis of Compound X2 >

Into a reactor, 6.1 g of Compound X1, 24.4 g of dimethyl sulfoxide (DMSO), 2.4 g of lithium chloride and 3.27 g of water were put and stirred in a nitrogen atmosphere at 180° C. After completion of the reaction, hydrochloric acid was added, an organic phase was recovered, and the recovered organic phase was concentrated by an evaporator and subjected to silica gel column chromatography to obtain 4.0 g of Compound X2.

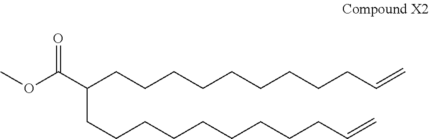 [SMILES: C=CCCCCCCCCCC(CCCCCCCCCC=C)C(=O)OC]

### <Synthesis of Compound X3>

Into a reactor, 2.0 g of Compound X2, 20.0 g of dehydrated tetrahydrofuran (THF), 6.6 mL of 2.0M lithium diisopropylamide (THF solution) and 3.1 g of 11-bromo-1-undecene (CH₂═CH(CH₂)₉—Br) were put and stirred in a nitrogen atmosphere at about −70° C. After completion of the reaction, hydrochloric acid was added, an organic phase was recovered, and the recovered organic phase was concentrated by an evaporator and subjected to silica gel column chromatography to obtain 2.1 g of Compound X3.

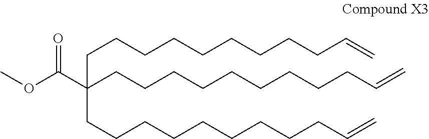 [SMILES: C=CCCCCCCCCCC(CCCCCCCCCC=C)(CCCCCCCCCC=C)C(=O)OC]

### <Synthesis of Compound X4>

Into a reactor, 0.89 g of lithium aluminum hydride and 25 g of dehydrated THF were put and stirred in a nitrogen atmosphere at 0° C. Then, 2.5 g of Compound X3 was put and stirred in a nitrogen atmosphere at 0° C. After completion of the reaction, water and 1M sodium hydroxide were put, an organic phase was recovered, and the recovered organic phase was concentrated by an evaporator and subjected to silica gel column chromatography to obtain 2.0 g of Compound X4.

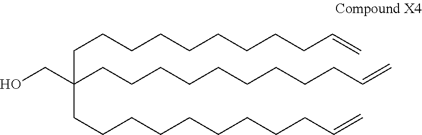 [SMILES: C=CCCCCCCCCCC(CO)(CCCCCCCCCC=C)CCCCCCCCCC=C]

### <Synthesis of Compound X5>

Using the above Compound X4, in accordance with the method in Examples 1 and 2 of WO2021/054413, the following Compound X5 was obtained.

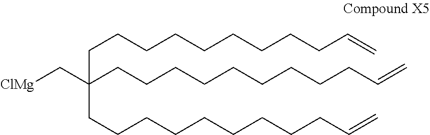

### <Synthesis of Compound A1-1>

1.03 g of the following Compound B1-1 was suspended in 5 ml of dehydrated THF, 0.0025 g of copper chloride was added and stirred at room temperature (25° C.). To the mixed solution, 1.15 g of the above Compound X5 adjusted to 17 mass % was slowly added dropwise, followed by stirring at 55° C. The mixed solution was cooled to room temperature (25° C.), water was added, extraction was conducted with 5 mL of -3000 (manufactured by AGC Inc., ASAHIKLIN (registered trademark) AE-3000), and sodium sulfate was added. After filtration, the solvent was distilled off. By conducting flash column chromatography with silica gel, a mixture containing Compound A1-1 was obtained. It was confirmed by NMR measurement that Compound A1-1 was obtained with a selectivity of 79%. In the formula, the average of the number n of repeating units is 13.

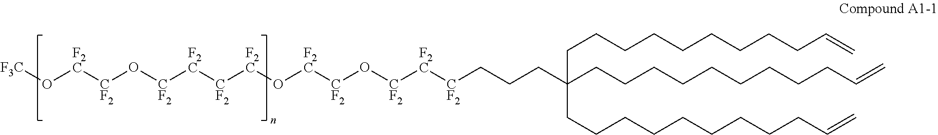

### Synthesis of Compound B1-1

Compound B1-1 was obtained in accordance with the method described in Example 11 of WO2013/121984.

CF₃—(OCF₂CF₂-OCF₂CF₂CF₂CF₂)ₙ(OCF₂CF₂)—OCF₂CF₂CF₂—CH₂CH₂I (B1-1)

The average of the number n of repeating units is 13.

### Synthesis of Compound B1-2

Into a flask, 3.0 g of the above Compound B1-1, 22 mg of azobisisobutyronitrile, 6.0 g of 1,3-bis(trifluoromethyl)benzene and 574 mg of tributyltin hydride were put and stirred at 70° C. for 2 hours. After the mixture was cooled to room temperature (25° C.), the obtained reaction solution was purified by silica gel column chromatography to obtain 2.3 g of Compound B1-2.

NMR spectrum data of Compound B1-2;
¹H-NMR (400 MHz, Chloroform-d) δ (ppm): 2.30 to 2.13 (m, 2H), 1.27 (t, J=7.1 Hz, 3H)
¹⁹F-NMR (376 MHz, Chloroform-d) δ (ppm): −55.25, −82.83, −88.06, −90.16 (d, J=8.1 Hz), −116.52, −125.26, −126.59.

CF₃—(OCF₂CF₂—OCF₂CF₂CF₂CF₂)ₙ(OCF₂CF₂)—OCF₂CF₂CF₂—CH₂CH₃ (B1-2)

The average of the number n of repeating units is 13.

### Synthesis of Compound B2-1

In an argon atmosphere, 3.0 g of the above Compound B1-1, 104 mg of metal copper and 3.0 mL of DMSO were put into a flask and stirred at 150° C. for 16 hours.

After the obtained reaction solution was cooled to room temperature (25° C.), it was purified by silica gel column chromatography to obtain 2.1 g of Compound B2-1. In the formula, the average of the number n of repeating units is 13.

NMR spectrum data of Compound B2-1;
¹H-NMR (400 MHz, Chloroform-d) δ (ppm): 2.31 to 2.14 (m, 4H), 1.91 to 1.78 (m, 4H)
¹⁹F-NMR (376 MHz, Chloroform-d) δ (ppm): −55.25, −82.83, −88.06, −90.16 (d, J=8.1 Hz), −114.33, −125.26, −126.59.

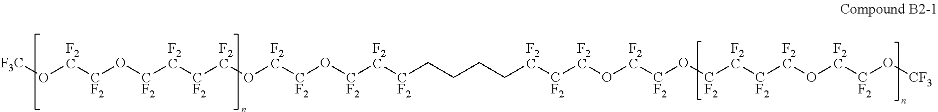

### Synthesis of Compound C1-1

### <Synthesis of Compound Y1>

60.0 g of diethyl diallyl malonate, 23.7g (559 mmol) of lithium chloride, 6.45 g (360 mmol) of water and 263 g of DMSO were added and stirred at 160° C. After the mixture was cooled to room temperature (25° C.), water was added and extraction with ethyl acetate was conducted. Hexane was added to an organic layer, and the organic layer was washed with saturated salt solution and dried over sodium sulfate. After filtration, the solvent was distilled off to obtain 39.5 g of the following Compound Y1.

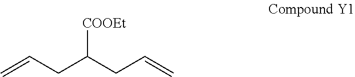

wherein Et is an ethyl group.

NMR spectrum data of Compound Y1;
¹H-NMR (400 MHz, Chloroform-d) δ (ppm): (ddt, J=17.1, 10.1, 7.0 Hz, 2H), 5.06 to 4.94 (m, 4H), 4.09 (q, J=7.1 Hz, 2H), 2.47 (ddd, J=14.0, 8.0, 6.1 Hz, 1H), 2.33 (dt, J=14.9, 7.5 Hz, 2H), 2.22 (dt, J=14.1, 6.5 Hz, 2H), 1.21 (t, J=7.1 Hz, 3H).

### <Synthesis of Compound Y2>

260 mL of THF and 29.8 mL of diisopropylamine were added, and the solution was cooled to −78° C. 96.6 mL of n-butyllithium hexane solution (2.76 M) was added and warmed to 0° C. After stirring, the mixture was cooled to −78° C. to prepare a THF solution of lithium diisopropylamide (LDA). 39.5 g of the above Compound Y1 was added to the THE solution and stirred, and 24.1 mL of allyl bromide was added. The mixture was warmed to 0° C., 100 mL of 1M hydrochloric acid was added, and THF was distilled off under reduced pressure. Extraction with dichloromethane was conducted, and sodium sulfate was added. After filtration, the solvent was distilled off, and flash column chromatography with silica gel was conducted to obtain 45.0 g of Compound Y2.

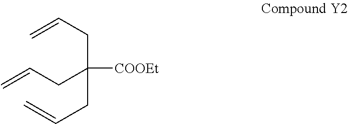

wherein Et is an ethyl group.

NMR spectrum data of Compound Y2;
¹H-NMR (400 MHz, Chloroform-d) δ (ppm): 5.74 to 5.62 (m, 3H), 5.04 (dd, J=13.6, 1.9 Hz, 6H), 4.10 (q, J=7.1 Hz, 2H), 2.29 (d, J=7.4 Hz, 6H), 1.22 (t, J=7.1 Hz, 3H).

### <Synthesis of Compound Y3>

45.0 g of the above Compound Y2 was dissolved in 620 mL of THF and cooled to 0° C. 104 mL of a THF solution of lithium aluminum hydride was added and stirred. Water and a 15% aqueous sodium hydroxide solution were added, and the mixture was stirred at room temperature (25° C.) and diluted with dichloromethane. After filtration, the solvent was distilled off, and flash column chromatography with silica gel was conducted to obtain 31.3 g of the following Compound Y3.

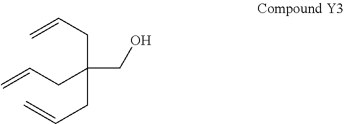

NMR spectrum data of Compound Y3;
¹H-NMR (400 MHz, Chloroform-d) δ (ppm): 5.90 to 5.76 (m, 3H), 5.10 to 5.02 (m, 6H), 3.38 (s, 2H), 2.03 (dt, J=7.5, 1.2 Hz, 6H), 1.45 (s, 1H).

### <Synthesis of Compound Y4>

380 mL of acetonitrile, 31.3 g of the above Compound Y3, 64.3 g of triphenylphosphine and 33.9 g of carbon tetrachloride were added and stirred at 90° C. After concentration, ethyl acetate and hexane were added and stirred. After filtration, the filtrate was concentrated and distilled to obtain 28.2 g of the following Compound Y4.

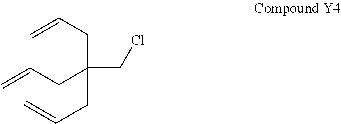

NMR spectrum data of Compound Y4;
¹H-NMR (400 MHz, Chloroform-d) δ (ppm): 5.83 to 5.67 (m, 3H), 5.16 to 5.01 (m, 6H), 3.32 (s, 2H), 2.05 (dt, J=7.5, 1.1 Hz, 6H).

### <Synthesis of Compound Y5>

35 mL of THF and 0.180 g of iodide were added to 2.36 g of magnesium and stirred at room temperature (25° C.). 14.0 g of the above Compound Y4 and 35 mL of THF were added and subjected to reflux with heating to obtain a solution (0.80M) of the following Compound Y5.

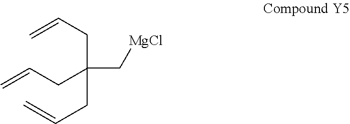

NMR spectrum data of Compound Y5;
¹H-NMR (400 MHz, Chloroform-d) δ (ppm): 5.88 (m, 3H), 5.11 (m, 6h), 1.85 (m, 6h), 1.22 (s, 2h).

### <Synthesis of Compound C1-1>

1.03 g of the above Compound B1-1 was suspended in 5 mL of dehydrated THF, 0.0025 g of copper chloride was added and stirred at room temperature (25° C.). To the mixed solution, 0.31 g of the above Compound Y5 adjusted to 17 mass % was slowly added dropwise and stirred at 55° C. The mixed solution was cooled to room temperature (25° C.), water was added, extraction with AE-3000 was conducted, and sodium sulfate was added. After filtration, the solvent was distilled off. Flash column chromatography with silica gel was conducted to obtain a mixture containing Compound C1-1. It was confirmed by NMR measurement that Compound C1-1 was obtained with a selectivity of 84%. In the formula, the average of the number n of repeating units was 13.

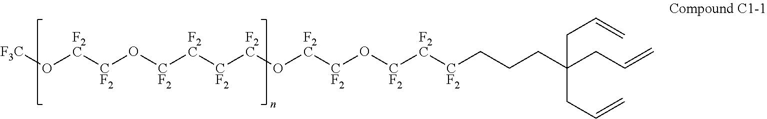

NMR spectrum data of Compound C1-1;
¹H-NMR (400 MHz, Chloroform-d) δ (ppm): 5.80 (ddt, J=20.3, 9.3, 7.4 Hz, 3H), 5.01 (dd, J=13.5, 1.7 Hz, 6H), 2.13 to 2.01 (m, 2H), 1.97 (d, J=7.5 Hz, 6H), 1.67 to 1.55 (m, 2H), 1.27 to 1.18 (m, 2H).
¹⁹F-NMR (376 MHz, Chloroform-d) δ (ppm): −55.25, −82.83, −88.06, −90.16 (d, J=8.1 Hz), −114.18, −125.26, −126.59.

### Example 1

1 g of a mixture containing Compound A1-1 (16 mol %), Compound B1-1 (10 mol %), Compound B1-2 (17 mol %) and Compound B2-1 (57 mol %) was prepared and dissolved in 1 g of AC-6000 to obtain reference composition X for extraction.

Hexane (10 g) as an extraction solvent was added to the reference composition X, and the obtained mixture was stirred at room temperature (23° C.) for 30 minutes. After stirring, the mixture was left at rest for 1 hour to separate into two layers, and the upper layer was recovered. Hexane and AC-6000 in the recovered upper layer were distilled off under reduced pressure to obtain sample 1 for purity measurement.

The concentrations (mol %) of the compounds contained in sample 1 were measured by ¹H-NMR and ¹⁹F-NMR. The results are shown in Table 1.

The concentrations mean the concentrations (mol %) of the respective compounds to the total amount (100 mol %) of the fluorinated ether compounds contained in the sample. The same applies to the following Examples

### Examples 2 and 3

In the same manner as in Example 1 except that the solvent as identified in Table 1 was used instead of hexane as the extraction solvent, the concentrations (mol %) of the respective compounds contained in each of samples in Examples 2 and 3 were measured. The results are shown in Table 1.

### Example 4

In the same manner as in Example 1 except that AE-3000 was used instead of hexane as the extraction solvent, the mixture after stirring was left at rest, however, the mixture was not separated into two layers.

The concentrations (mol %) of the respective components contained in the mixture were measured in the same manner as in Example 1 and found to be the same as the concentrations of the compounds contained in the reference composition X.

TABLE 1

| | | Reference | | | | |
|---|---|---|---|---|---|---|
| | | composition X | Example 1 | Example 2 | Example 3 | Example 4 |
| Extraction | Type | — | Hexane | Acetone | Dichloro- | AE-3000 |
| solvent | | | | | methane | |
| | Relative | — | 1.8 | 20.6 | 8.9 | 6.6 |
| | permittivity | | | | | |
| Concentration | Compound | 16 | 83 | 58 | 27 | 16 |
| (mol %) | A1-1 | | | | | |
| | Compound | 10 | 9 | 0 | 3 | 10 |
| | B1-1 | | | | | |
| | Compound | 17 | 7 | 35 | 12 | 17 |
| | B1-2 | | | | | |
| | Compound | 57 | 1 | 7 | 58 | 57 |
| | B2-1 | | | | | |

As shown in Table 1, it was confirmed that in extraction of the first component (Compound A1-1) from the composition containing the first component and the second component, by using the organic solvent containing no fluorine atom as the extraction solvent, the first component can efficiently be extracted (Examples 1 to 3).

### Example 5

1 g of a mixture containing Compound C1-1 (25 mol %), Compound B1-1 (7 mol %), Compound B1-2 (27 mol %) and Compound B2-1 (41 mol %) was prepared and dissolved in 1 g of AC-6000 to obtain reference composition Y for extraction.

10 g of hexane as an extraction solvent was added to the reference composition Y, and the obtained mixture was stirred at room temperature (23° C.) for 30 minutes. After stirring, the mixture was left at rest for 1 hour to separate into two layers, and the upper layer was recovered. Hexane and AC-6000 in the recovered upper layer were distilled off under reduced pressure to obtain sample 5 for purity measurement.

In the same manner as in Example 1 except that the sample 5 was used instead of the sample 1, the concentrations (mol %) of the respective compounds contained in the sample in Example 5 were measured. The results are shown in Table 2.

### Example 6

In the same manner as in Example 5 except that AE-3000 was used instead of hexane as the extraction solvent, the mixture after stirring was left at rest, however, the mixture was not separated into two layers.

The concentrations (mol %) of the respective components contained in the mixture were measured in the same manner as in Example 5 and found to be the same as the concentrations of the compounds contained in the reference composition Y.

TABLE 2

| | | Reference | | |
|---|---|---|---|---|
| | | composition | Example | Example |
| | | Y | 5 | 6 |
| Extraction solvent | | — | Hexane | AE-3000 |
| Concentration | Compound | 25 | 34 | 25 |
| (mol %) | C1-1 | | | |
| | Compound | 7 | 25 | 7 |
| | B1-1 | | | |
| | Compound | 27 | 37 | 27 |
| | B1-2 | | | |
| | Compound | 41 | 4 | 41 |
| | B2-1 | | | |

As shown in Table 2, in a case where Compound C1-1 (compound other than the above described Compound A1, Compound A2 and Compound A3) was used as the fluorinated ether compound to be extracted, instead of Compound A1-1, Compound C1-1 to be extracted could not efficiently be extracted even by using hexane as the extraction solvent with which the fluorinated ether compound to be extracted could be extracted most efficiently among Examples 1 to 4 (Example 5).

This application is a continuation of PCT Application No. PCT/JP2023/036912, filed on Oct. 11, 2023, which is based upon and claims the benefit of priority from Japanese Patent Application No. 2022-164649 filed on Oct. 13, 2022. The contents of those applications are incorporated herein by reference in their entireties.

What is claimed is:

1. A method for extracting a fluorinated ether compound, which comprises extracting a first component from a composition, using an organic solvent containing no fluorine atom, the composition containing:
the first component composed of at least one fluorinated ether compound selected from the group consisting of a compound represented by the formula (A1), a compound represented by the formula (A2) and a compound represented by the formula (A3), and
a second component composed of at least one fluorinated ether compound selected from the group consisting of a compound represented by the formula (B1) and a compound represented by the formula (B2),
[R^{f1}—(OR^{f11})_{y1}—O—R¹]ⱼ-L¹-(R¹¹-T¹¹)ₓ₁ (A1)
(T³¹-R³)ₓ₃-L³-R³—(OR^{f12})_{y2}—O—R²-L²-(R²¹-T²¹)ₓ₂ (A2)
Q¹[-(OR^{f13})_{y3}—O—R⁴-L⁴-(R⁴¹-T⁴¹)ₓ₄]ᵣ₁ (A3)
R^{f14}—(OR^{f15})_{y4}—O—R⁵-R^{b1} (B1)
R^{f19}—(OR^{f18})_{y6}—O—R⁷-L^{b1}-R⁶—O—(R^{f16}O)_{y5}—R^{f17} (B2)
in the formula (A1),
R^{f1} is a C₁₋₂₀ fluoroalkyl group, and when there is a plurality of R^{f1}, the plurality of R^{f1} may be the same or different from each other,
R^{f11} is a C₁₋₆ fluoroalkylene group, and when there is a plurality of R^{f11}, the plurality of R^{f11} may be the same or different from each other,
R¹ is an alkylene group or a fluoroalkylene group, and when there is a plurality of R¹, the plurality of R¹ may be the same or different from each other,
L¹ is a single bond or a (j+x1)-valent group which may have N, O, S or Si and may have a branch point, and in which atoms bonded to R¹ and R¹¹ are each independently a N, O, S, Si atom, a carbon atom constituting the branch point, a hydroxy group or a carbon atom having an oxo group (═O),
R¹¹ is an alkylene group in which an atom bonded to L¹ may be an etheric oxygen atom, and which may have an etheric oxygen atom between carbon atoms, and when there is a plurality of R¹¹, the plurality of R¹¹ may be the same or different from each other,
T¹¹ is —CH═CH₂ or —SiR^{a11}_{z11}R^{a12}_{3-z11}, and when there is a plurality of T¹¹, the plurality of T¹¹ may be the same or different from each other,
in a case where T¹¹ is —CH═CH₂, at least one R¹¹ has a carbon chain having 2 or more carbon atoms linked to each other, and the carbon chain is linked to T¹¹,
in a case where T¹¹ is —SiR^{a11}_{z11}R^{a12}_{3-z11}, at least one R¹¹ has a carbon chain having 4 or more carbon atoms linked to one another,
R^{a11} is a hydroxy group or a hydrolysable group, and when there is a plurality of R^{a11}, the plurality of R^{a11} may be the same or different from each other,
R^{a12} is a non-hydrolysable group, and when there is a plurality of R^{a12}, the plurality of R^{a12} may be the same or different from each other,
j is an integer of 1 or more, and in a case where j is 1, y1 is an integer of 2 or more, or R¹ is a fluoroalkylene group,
z11 is an integer of 1 to 3,
x1 is an integer of 1 or more, and
y1 is an integer of 1 or more, and when there is a plurality of y1, the plurality of y1 may be the same or different from each other,
in the formula (A2),
R^{f12} is a C₁₋₆ fluoroalkylene group, and when there is a plurality of R^{f12}, the plurality of R^{f12} may be the same or different from each other,
R² and R³ are each independently an alkylene group or a fluoroalkylene group,
L² is a single bond or a (1+x2)-valent group which may have N, O, S or Si and may have a branch point, and in which atoms bonded to R² and R²¹ are each independently a N, O, S, Si atom, a carbon atom constituting the branch point, a hydroxy group or a carbon atom having an oxo group (═O),
L3 is a single bond or a (1+x3)-valent group which may have N, O, S or Si and may have a branch point, and in which atoms bonded to R³ and R³¹ are each independently a N, O, S, Si atom, a carbon atom constituting the branch point, a hydroxy group or a carbon atom having an oxo group (═O),
R²¹ is an alkylene group in which an atom adjacent to L² may be an etheric oxygen atom, and which may have an etheric oxygen atom between carbon atoms, and when there is a plurality of R²¹, the plurality of R²¹ may be the same or different from each other,
R³¹ is an alkylene group in which an atom adjacent to L³ may be an etheric oxygen atom, and which may have an etheric oxygen atom between carbon atoms, and when there is a plurality of R³¹, the plurality of R³¹ may be the same or different from each other,
T²¹ and T³¹ are each independently —CH═CH₂ or —SiR^{a21}_{z21}R^{a22}_{3-z21}, when there is a plurality of T²¹, the plurality of T²¹ may be the same or different from each other, and when there is a plurality of T³¹, the plurality of T³¹ may be the same or different from each other,
in a case where T²¹ is —CH═CH₂, at least one R²¹ has a carbon chain having 2 or more carbon atoms linked to each other, and the carbon chain is linked to T²¹,
in a case where T²¹ is —SiR^{a21}_{z21}R^{a22}_{3-z21}, at least one R²¹ has a carbon chain having 4 or more carbon atoms linked to one another,
in a case where T³¹ is —CH═CH₂, at least one R³¹ has a carbon chain having 2 or more carbon atoms linked to each other, and the carbon chain is linked to T³¹,
in a case where T³¹ is —SiR^{a21}_{z21}R^{a22}_{3-z21}, at least one R³¹ has a carbon chain having 4 or more carbon atoms linked to one another,
R^{a21} is a hydroxy group or a hydrolysable group, and when there is a plurality of R^{a21}, the plurality of R^{a21} may be the same or different from each other,
R^{a22} is a non-hydrolysable group, and when there is a plurality of R^{a22}, the plurality of R^{a22} may be the same or different from each other,
z21 is an integer of 1 to 3,
x2 and x3 are each independently an integer of 1 or more, and
y2 is an integer of 1 or more, and in a case where y2 is 1, R² is a fluoroalkylene group,
in the formula (A3),
Q¹ is a r1-valent group having a branch point,
R^{f13} is a C₁₋₆ fluoroalkylene group, and the plurality of R^{f13} may be the same or different from each other,
R⁴ is each independently an alkylene group or a fluoroalkylene group, and the plurality of R⁴ may be the same or different from each other,
L⁴ is a single bond or a (1+x4)-valent group which may have N, O, S or Si and may have a branch point, and in which atoms bonded to R⁴ and R⁴¹ are each independently a N, O, S, Si atom, a carbon atom constituting the branch point, a hydroxy group or a carbon atom having an oxo group (═O), and the plurality of L⁴ may be the same or different from each other,
R⁴¹ is an alkylene group in which an atom adjacent to L⁴ may be an etheric oxygen atom, and which may have an etheric oxygen atom between carbon atoms, and the plurality of R⁴¹ may be the same or different from each other,
T⁴¹ is —CH═CH₂ of —SiR^{a41}_{z41}R^{a42}_{3-z41}, and the plurality of T⁴¹ may be the same or different from each other,
in a case where T⁴¹ is —CH═CH₂, at least one R⁴¹ has a carbon chain having 2 or more carbon atoms linked to each other, and the carbon chain is linked to T⁴¹,
in a case where T⁴¹ is —SiR^{a41}_{z41}R^{a42}_{3-z41}, at least one R⁴¹ has a carbon chain having 4 or more carbon atoms linked to one another,
R^{a41} is a hydroxy group or a hydrolysable group, and the plurality of R^{a41} may be the same or different from each other,
R^{a42} is a non-hydrolysable group, and when there is a plurality of R^{a42}, the plurality of R^{a42} may be the same or different from each other,
z41 is an integer of 1 to 3,
x4 is an integer of 1 or more, and when there is a plurality of x4, the plurality of x4 may be the same or different from each other,
y3 is an integer of 1 or more, and when there is a plurality of y3, the plurality of y3 may be the same or different from each other, and
r1 is 3 or 4,
in the formula (B1),
R^{f14} is a C₁₋₂₀ fluoroalkyl group,
R^{f15} is a C₁₋₆ fluoroalkylene group, and when there is a plurality of R^{f15}, the plurality of R^{f15} may be the same or different from each other,
R⁵ is an alkylene group which may have a substituent,
R^{b1} is a hydrogen atom, a chlorine atom, a bromine atom, or an iodine atom, and
y4 is an integer of 1 or more,
in the formula (B2),
R^{f17} and R^{f19} are each independently a C₁₋₂₀ fluoroalkyl group,
R^{f16} and R^{f18} are each independently a C₁₋₆ fluoroalkylene group, when there is a plurality of R^{f16}, the plurality of R^{f16} may be the same or different from each other, and when there is a plurality of R^{f18}, the plurality of R^{f18} may be the same or different from each other,
R⁶ and R⁷ are each independently an alkylene group which may have a substituent,
L^{b1} is a single bond or a bivalent linking group (excluding (OR^{f18})_{y7} and (R^{f16}O)_{y8}, y7 and y8 are each independently an integer of 1 or more), and
y5 and y6 are each independently an integer of 1 or more.

2. The fluorinated ether compound extraction method according to claim 1, wherein the organic solvent is a non-halogenated solvent.

3. The fluorinated ether compound extraction method according to claim 2, wherein the non-halogenated solvent has a relative permittivity of 30 or less.

4. The fluorinated ether compound extraction method according to claim 1, wherein the organic solvent is a hydrocarbon-based solvent.

5. The fluorinated ether compound extraction method according to claim 1, wherein in the formula (A1), in a case where T¹¹ is —CH═CH₂, at least one R¹¹ has a carbon chain having 4 or more carbon atoms linked to one another and the carbon chain is linked to T¹¹, and in the formula (A1), in a case where T¹¹ is —SiR^{a11}_{z11}R^{a12}_{3-z11}, at least one R¹¹ has a carbon chain having 6 or more carbon atoms linked to one another,
in the formula (A2), in a case where T²¹ is —CH═CH₂, at least one R²¹ has a carbon chain having 4 or more carbon atoms linked to one another and the carbon chain is linked to T²¹, and in the formula (A2), in a case where T²¹ is —SiR^{a21}_{z21}R^{a22}_{3-z21}, at least one R²¹ has a carbon chain having 6 or more carbon atoms linked to one another,
in the formula (A2), in a case where T³¹ is —CH═CH₂, at least one R³¹ has a carbon chain having 4 or more carbon atoms linked to one another and the carbon chain is linked to T³¹, and in the formula (A2), in a case where T³¹ is —SiR^{a21}_{z21}R^{a22}_{3-z21}, at least one R³¹ has a carbon chain having 6 or more carbon atoms linked to one another, and
in the formula (A3), in a case where T⁴¹ is —CH═CH₂, at least one R⁴¹ has a carbon chain having 4 or more carbon atoms linked to one another and the carbon chain is linked to T⁴¹, and in the formula (A3), in a case where T⁴¹ is —SiR^{a41}_{z41}R^{a42}_{3-z41}, at least one R⁴¹ has a carbon chain having 6 or more carbon atoms linked to one another.

6. The fluorinated ether compound extraction method according to claim 1, wherein the group represented by (OR^{f11})_{y1} has a molecular weight of 2,000 to 20,000,
the group represented by (OR^{f12})_{y2} has a molecular weight of 2,000 to 20,000, and
the group represented by (OR^{f13})_{y3} has a molecular weight of 2,000 to 20,000.

7. The fluorinated ether compound extraction method according to claim 1, wherein
a ratio of a molecular weight of the group represented by [R^{f1}—(OR^{f11})_{y1}—O—R¹]ⱼ to a molecular weight of the group represented by L¹-(R¹¹-T¹¹)ₓ₁ is 13% or more,
a ratio of a molecular weight of the group represented by R³—(OR^{f12})_{y2}—O—R² to a sum of a molecular weight of the group represented by L²-(R²¹-T²¹)ₓ₂ and a molecular weight of the group represented by (T³¹-R³¹)ₓ₃-L³ is 13% or more, and
a ratio of a sum of a sum of molecular weights of r1 pieces of groups represented by (OR^{f13})_{y3}—O—R⁴ and a molecular weight of the group represented by Q¹, to a sum of molecular weights of r1 pieces of groups represented by L⁴-(R⁴¹-T⁴¹)ₓ₄, is 13% or more.

8. The fluorinated ether compound extraction method according to claim 1, wherein
in the formula (B1), R⁵ is a fluoroalkylene group, and
in the formula (B2), both R⁶ and R⁷ are a fluoroalkylene group, and L^{b1} is a single bond.

9. The fluorinated ether compound extraction method according to claim 8, wherein
in the formula (B1), a number of carbon atoms in the fluoroalkylene group represented by R⁵ is 1 to 20, and
in the formula (B2), a total number of carbon atoms in the fluoroalkylene group represented by R⁶ and the fluoroalkylene group represented by R⁷ is 2 to 40.
